(19) United States
(12) Patent Application Publication
(10) Pub. No.: US 2026/0283605 A1
(43) Pub. Date: Sep. 24, 2026

(54) REMOVABLE HANDLE DEVICE FOR ONE OR MORE ENDOLUMINAL ACCESSORIES

(71) Applicant: Endovision Foundation, Sacramento, CA (US)

(72) Inventors: Kenneth F. Binmoeller, San Francisco, CA (US); John P. Lunsford, San Carlos, CA (US); Hoang Phan, Milpitas, CA (US); Fiona Sander, Los Altos Hills, CA (US)

(21) Appl. No.: 19/086,995

(22) Filed: Mar. 21, 2025

(51) Int. Cl. A61B 17/00; A61B 1/018

(52) U.S. Cl. CPC A61B 17/00234; A61B 1/018; A61B 2017/0034; A61B 2017/00438; A61B 2017/0046; A61B 2017/00738

(57) ABSTRACT

A removable handle for an endoscope-compatible accessory has a first end opposite a second end, a groove for receiving at least a portion of a manipulatable element of an endoluminal accessory, and a coupling element on the second end for reversibly coupling to the endoluminal accessory. A clamping portion is slidable along the shaft and reversibly clamp to the manipulatable element of the endoluminal accessory, wherein a clamped state of the clamping portion allows manipulation of the manipulatable element. The clamping portion may include finger loops, a channel for receiving an elongate body, or an electrode for current application. A rotatable lock mechanism at the second end secures or releases the endoluminal accessory. The handle enables controlled actuation of the accessory within an endoscope's working channel while permitting removal and reinsertion without disturbing the accessory's position.

### INCORPORATION BY REFERENCE

All publications and patent applications mentioned in this specification are herein incorporated by reference in their entirety, as if each individual publication or patent application was specifically and individually indicated to be incorporated by reference in its entirety.

### TECHNICAL FIELD

This disclosure relates generally to the field of medical equipment, and more specifically, to the field of endoluminal tools. Described herein are systems and methods for a removable handle device for one or more endoluminal accessories in endoluminal instruments.

### BACKGROUND

Endoscopic procedures are medical procedures in which one or more medical cameras (e.g., endoscopes) are inserted into a patient's anatomy to view one or more target internal areas. Often one or more additional medical tools (e.g., endoluminal accessories such as snares, cutting accessories including, but not limited to, knives, baskets, forceps, etc.) may be subsequently inserted to operate on the tissue internal to the patient. In some examples, the endoscopes and endoluminal accessories enter the body through natural cavities and entryways (e.g., the mouth or anus of the gastrointestinal tract). In laparoscopic examples, a surgeon may make an incision in the patient's tissue (e.g., through soft tissue of the abdomen) to access the target areas with endoscopes (e.g., a laparoscope) or other endoluminal instruments. Due to the long and often circuitous paths that endoscopes and endoluminal instruments traverse in the body, endoscopes and endoluminal instruments often include long, flexible shafts, cables and/or tethers that connect controls or handles that are external to the body to the active component (e.g., camera lens, snare loop, etc.) inside the body.

### SUMMARY

In some aspects, the techniques described herein relate to a removable handle for an endoscope compatible accessory, including: a shaft having: a first end opposite a second end, a groove configured to receive at least a portion of a manipulatable element of an endoluminal accessory, and a coupling element on the second end configured to reversibly couple to the endoluminal accessory; and a clamping portion configured to slide along the shaft and reversibly clamp to the manipulatable element of the endoluminal accessory, wherein a clamped state of the clamping portion is configured to allow manipulation of the manipulatable element by the clamping portion.

In some aspects, the techniques described herein relate to a method of removing a handle from an endoluminal accessory including inserting at least a portion of a first endoluminal accessory into a patient; interacting with a first tissue portion with the first endoluminal accessory; and removing a handle of the first endoluminal accessory while maintaining the interaction with the first tissue portion.

In some aspects, the techniques described herein relate to a method of accessing a bodily region with two or more endoluminal accessories, including inserting at least a portion of a first endoluminal accessory into a lumen defined by a first endoluminal instrument; interacting with a first tissue portion with the first endoluminal accessory; removing a handle of the first endoluminal accessory while maintaining the interaction with the first tissue portion; removing the first endoluminal instrument while maintaining the interaction with the first tissue portion; reinserting the first endoluminal instrument parallel to the first endoluminal accessory; and inserting at least a portion of a second endoluminal accessory into the lumen defined by the first endoluminal instrument.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various embodiments, with reference made to the accompanying drawings.

FIG. 1 illustrates a handle of a snare accessory of the prior art.

FIG. 2A illustrates a cross-sectional view of an embodiment of a removable handle of an endoluminal accessory for slidably traversing at least a portion of a length of a shaft in a first position and an elongate body to engage at least a portion of a manipulatable element.

FIG. 2B illustrates a cross-sectional view of the removable handle of FIG. 2A oriented in a second position.

FIG. 3A illustrates an exploded view of an embodiment of a clamping portion of a removable handle for engaging and retaining at least a portion of a snare.

FIG. 3B illustrates an exploded view of an embodiment with the clamping portion of FIG. 3A having a guide structure that may be received by a channel and a coupling element disposed at an end of the shaft of a removable handle.

FIG. 3C illustrates a cross-sectional view of an embodiment of the guide structure of FIG. 3B with a clamping eyelet.

FIG. 3D illustrates a cross-sectional view of an embodiment of the clamping eyelet of FIG. 3C positioned within an aperture of the shaft.

FIG. 3E illustrates a front sectional view of the clamping eyelet of FIG. 3D partially retaining the manipulatable element.

FIG. 3F illustrates a perspective sectional view of the clamping eyelet of FIG. 3E partially retaining the manipulatable element.

FIG. 3G illustrates a schematic of an embodiment of a locking aperture.

FIG. 3H illustrates a schematic of an embodiment of an entry aperture.

FIG. 4A illustrates a cross-sectional view of an embodiment of a clamping portion of a removable handle.

FIG. 4B illustrates a cross-sectional view of a detail of an embodiment of a clamping portion of a removable handle.

FIG. 5A illustrates a perspective view of another embodiment of a coupling element with a rotatable lock oriented in a locked position to retain a sheath and connected to the shaft of a removable handle.

FIG. 5B illustrates a perspective view of the coupling element of FIG. 5A oriented in an unlocked position.

FIG. 6 illustrates a perspective view of the coupling element of FIG. 5A with a reduced diameter section.

FIG. 7 illustrates a perspective view the coupling element of FIG. 5A oriented in an unlocked position and retaining a portion of a sheath.

FIG. 8A illustrates a semi-transparent view of an embodiment of a coupling element.

FIG. 8B illustrates a cross-sectional view of an embodiment of a coupling element.

FIG. 9A illustrates a cross-sectional, exploded view of an embodiment of a removable handle.

FIG. 9B illustrates a zoomed-in, cross-sectional view of the distal portion of an embodiment of a removable handle.

FIG. 10A illustrates a cross-sectional view of an embodiment of a removable handle for modifying an endoluminal accessory.

FIG. 10B illustrates a detailed cross-sectional view of the sheath of circle B of FIG. 10A with an example of one or more attachment elements retaining a cable.

FIG. 10C illustrates a detailed cross-sectional view of the sheath of circle C of FIG. 10A with an example of a friction length with no friction receivers.

FIG. 10D illustrates a detailed cross-sectional view of the sheath of circle D of FIG. 10A with an example of a cable grip to be inserted around the cable and into the crimped hypotube having one or more crimps.

FIG. 10E illustrates a sectional cut-away view of an example cable grip of FIG. 10D with a distal end having one or more fingers

FIG. 11 illustrates an exploded view of an embodiment of the adapter of FIG. 10A with self-retaining features that may or may not be coupled with the removable handle.

FIG. 12 illustrates a flowchart of a method for removing the handle from an endoluminal accessory.

FIG. 13 illustrates a flowchart of an embodiment of a method for accessing a bodily region with two or more endoluminal accessories.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the claimed subject matter. Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

There is a need for a new and useful system and method for a removable handle for an endoscope compatible accessory. In particular, there is a need for systems, devices, and methods that allow a removable handle of an endoluminal accessory inserted through the working channel of an endoscope (or endoluminal instrument) or other delivery instrument to be removed. This enables the endoscope or delivery instrument to be withdrawn while keeping the accessory actuated. Additionally, there is a need for the removable handle to be reattached, allowing the endoluminal accessory to be de-actuated after the endoscope or delivery instrument is reinserted coaxially (i.e., other the endoluminal accessory) or parallel to the endoluminal accessory in a bodily lumen in which they are both inserted.

A handle as used herein may be a manipulatable feature of an instrument that may be used for a purpose, one or more purposes, or a plurality of purposes. For example, a handle may be used as an actuator, connector, gripping surface, etc. A handle may be used for precision, control, and ease of use. A handle may provide a grip to reduce slippage during a procedure. A handle may be textured or ergonomic to increase control. A handle may apply a controlled force. A handle may be used to manipulate tissues, cutting, or holding objects. A handle may provide fine motor control, for example during minimally invasive or laparoscopic procedures. A handle may be used to operate moving parts of an instrument (e.g., an opening or closing mechanism, a gripping mechanism, a suturing mechanism, a camera angle mechanism, a tool manipulation mechanism, etc.). A handle may allow for interchangeable tips and/or improve modularity of an instrument. A handle may be cleanable or sterilizable. A handle may be non-porous and/or smooth. A handle may provide feedback to a user.

Disclosed herein are accessories, devices, and related methods for a removable handle for use with an accessory that is inserted through an endoscope or other accessory delivery platform. The endoscope or other accessory delivery platform is inserted into an elongate cavity (e.g., a bodily lumen) defined by a structure, either a natural structure (e.g., defined by intestine, etc.) or an artificial structure (e.g., defined by a conduit such as a stent). In some embodiments, the devices herein include a clamping element that secures to a manipulatable element of the endoluminal accessory and a coupling element that secures to a shaft of the accessory such that an operator of the device may maneuver or activate the manipulatable element relative to the shaft of the accessory. In some embodiments, the clamping element and coupling element are reversible, allowing for the handle to be selectively attached, detached, and reattached according to the needs of an endoscopic procedure. For example, the removable handle of an endoluminal accessory may be removed, in some embodiments, to make space available for the removal of an endoscope while the endoluminal accessory maintains its grip or actuation state on target tissue during the time the removable handle is absent. In some embodiments, the device may further include friction elements between the manipulatable element, the handle, and/or the device lumen that prevent or reduce unintentional motion of the manipulatable element while or when the handle has been detached.

The device functions as a handle that is reversibly attachable or couplable to an accessory compatible with an endoscope, an accessory delivery platform, or an endoscopy procedure. The accessory is used for endoscopy procedures but can additionally, or alternatively, be used for any suitable applications, clinical or otherwise. The device can be adapted to function for any suitable accessory where a removable handle is desirable.

In some embodiments, the accessories, devices, and related methods described herein function to reversibly or removably attach or couple to an endoscope or other operational endoluminal accessory or instrument used during an endoscopy procedure, such as various surgical snares, forceps, baskets, ballons, etc. The removal of the handle, in some embodiments, during a procedure enables the removal of the endoscope, which can be exchanged coaxially for another endoluminal instrument such as an endoscope (e.g., thinner, different or enhanced optics), echoendoscope (added ultrasound imaging), or other coaxially insertable instrument (e.g., multichannel endoscopic access device or a flexible endoscopic access platform). As described elsewhere herein, the term “device” refers to a removable handle. The term “accessory” described a catheter-based tool that is introduced through the working channel of an endoscope. The term “instrument” encompasses any device capable of being inserted into a bodily lumen, including but not limited to, an endoscope or echoendoscope.

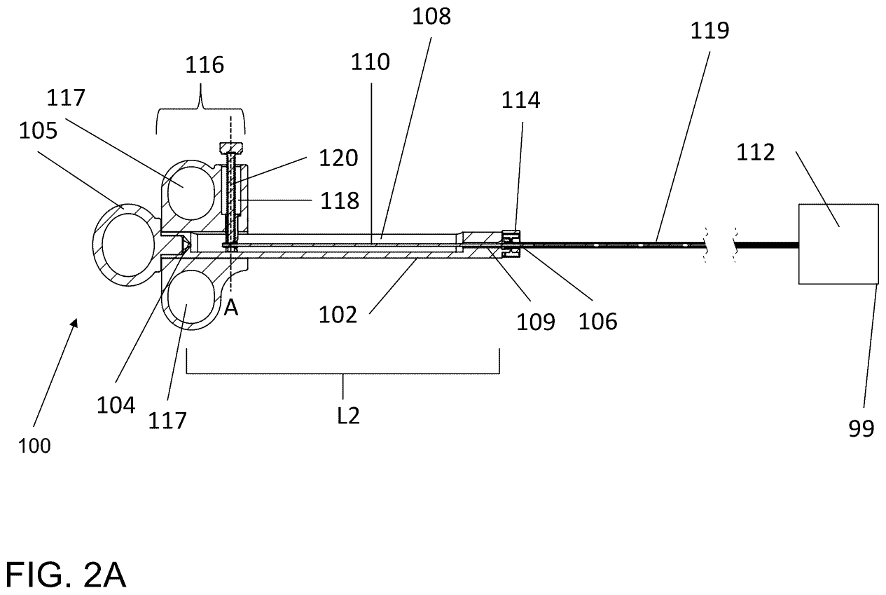

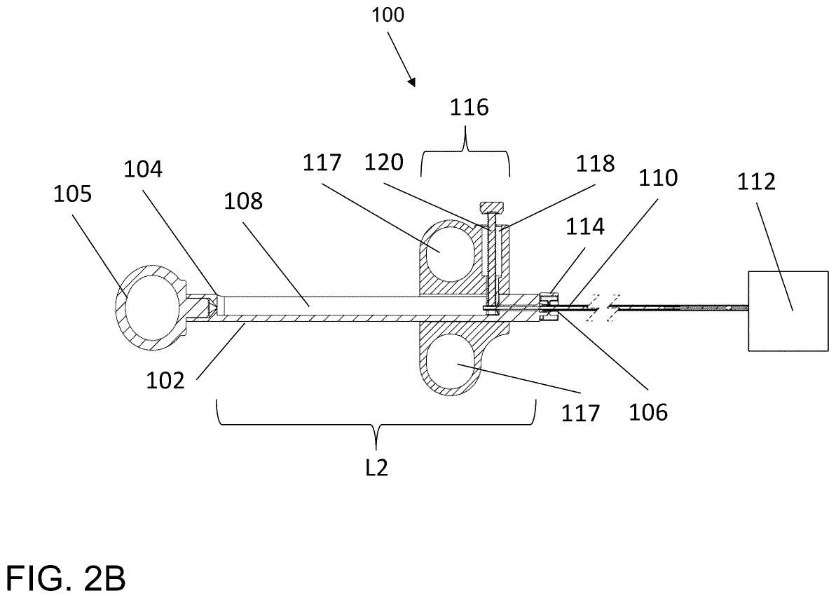

For example, endoscopes may be sufficiently durable to be considered pushable, which means that they may be delivered into the body of a patient by force from the proximal end without a previously installed guide channel, also called a working channel. Some medical tools and endoluminal accessories used during endoscopic procedures may be too flexible to be pushable. Such tools instead may benefit from navigation through a guide channel (e.g., of an endoscope, guide catheter, sheath, cannula, etc.) inserted first so that the accessory's working end (e.g., distal end 99 of the endoluminal accessory 112 of FIG. 2A) may safely traverse the length of the guide channel, also referred to as a working channel, to arrive at the operation site within the body. Various components of the endoluminal accessory, such as tethers, control lines, guide wires, or cables, which connect these tools to controls or handles exterior to the body and are herein referred to as the “working length” of the endoluminal accessory, occupy a limited interior volume of the working channel. In some embodiments, at least a portion of the working length of an endoluminal accessory is encased within a sheath or a tube-like device used to facilitate the insertion and/or exchange of catheters and other medical instruments into the body.

In some embodiments, the physical space and position occupied by a handle of a tool, a handle of an accessory, or other components produces procedural constraints on an operation. For example, a handle of a tool or an accessory exterior to the body may prohibit the passage of other accessories through the working channel and the removal of the endoscope from the body while the shaft of the tool or the accessory remains inserted within the body. As a result, in some embodiments, the tool or accessory stays in place with the distal end effector may remain attached to the body. In these situations, the tool or accessory is removed (undesirable or impractical or not feasible in some examples) or a second pushable endoluminal instrument (e.g., endoscope) is inserted to provide a new, second working channel (for which there may not be space or may present issues) to deliver a tool or accessory. For example, using a second pushable endoluminal instrument may be costly in supplies and time and may also be undesirable or impractical or not feasible due to anatomical constraints. Additionally, many prior art accessories, endoluminal instruments, and medical tools have features or aspects of their controls connected through their handles, making removal of the handle difficult if not impossible.

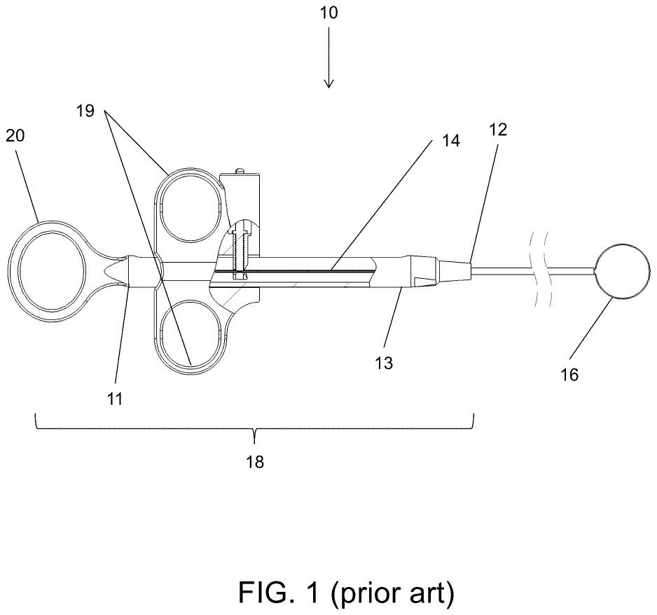

For example, as shown in FIG. 1, a prior art surgical snare 10 (e.g., a prior art endoluminal accessory) has a control wire 14 that adjusts the size of the snare loop 16 extending from a distal end 12. The control wire 14 runs through the handle 18 and connects to a finger loop 19 which slides along the shaft 13. Secured with a thumb in a thumb loop 20 positioned on a proximal end 11, the finger loops 19 are moved to actuate the control wire 14 and therefore actuate the snare loop 16. However, because the control wire 14 is irreversibly attached to the finger loops 19 in the handle 18, the surgical snare 10 remains as one continuous piece to retain operation of the snare loop 16. The handle 18 cannot be removed from the surgical snare 10 without destroying the functionality of the surgical snare 10 (i.e., the specified size of the snare loop 16) unless further, immensely impractical modifications are made to the surgical snare 10 during the operation.

The devices, accessories, and instruments described herein solve the above technical problems with technical solutions. For example, the devices described herein provide at least the technical solution of a reversible attachment mechanism that allows the detachment and reattachment of the handle from the endoluminal accessory without interfering (or only minimally interfering) with the operable state of the endoluminal accessory (e.g., snare size, forceps grip position, camera position, etc.) Once the handle is removed, an endoluminal instrument having the working channel may be removed over the working length of the accessory. By at least this technical solution, the endoluminal instrument may be removed and reinserted with an empty working channel available for additional endoluminal accessories (e.g., second, third, etc.) without disrupting or interfering with the operable state of the first endoluminal accessory.

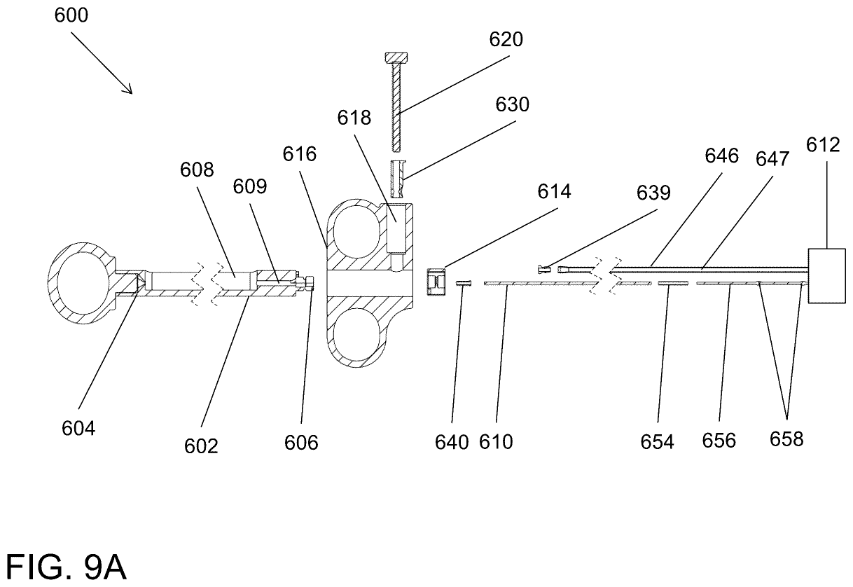

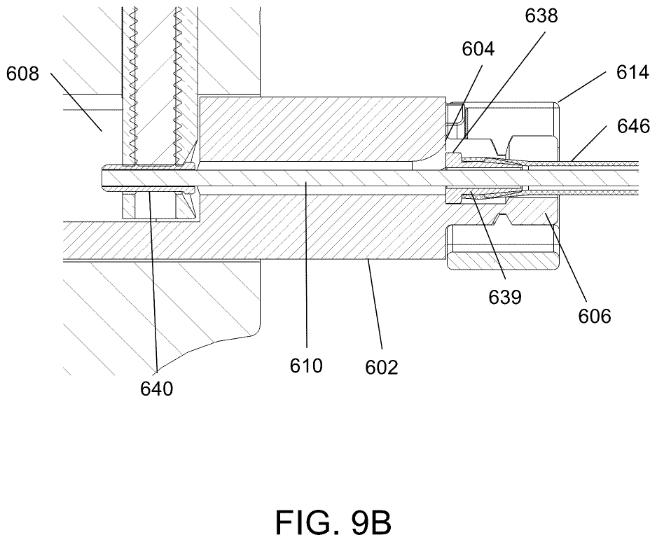

FIGS. 2A-2B show a cross-section of an embodiment of a removable handle 100 for endoluminal accessories. In some embodiments, the removable handle 100 includes a shaft 102 having a first end 104 (proximal end) opposite a second end 106 (distal end). In some embodiments, the shaft 102 (e.g., on the first end 104) may include at least a portion of a grip stabilizer 105 (e.g., a thumb loop as shown in the embodiment of FIG. 2A.) The shaft 102 may define a groove 108, in some embodiments, that may receive at least a portion of a manipulatable element 110 of an endoluminal accessory 112. In some embodiments, the manipulatable element 110 may enter the groove 108 through a lumen 109 of the second end 106. In some embodiments, the endoluminal accessory 112 may be, but is not limited to, a snare, a grasper, a pair of forceps, a clip applier, a loop ligation accessory (e.g., PolyLoop®), an injection needle, a retrieval accessory, a retrieval basket, an electrosurgical knife, a sphincterotome, an extraction balloon, a lithotripter, an intraluminal balloon, and/or a stent. For example, the manipulatable element 110 of a grasper may be an actuator shaft to close and open the grasper. A shaft is an elongated structural member for transmitting force or motion. In the context of an endoscopic accessory, the shaft may be rigid or flexible, including but not limited to, a flexible cable or catheter, to facilitate navigation through anatomical pathways while maintaining structural integrity to perform the intended function. For example, the manipulatable element 110 of a forceps may be an actuator shaft to close or open the forceps. For example, the manipulatable element 110 of a loop ligation accessory may be an actuator shaft to apply and secure a ligation accessory. For example, the manipulatable element 110 of a retrieval basket may be an actuator shaft to cinch closed or open the retrieval basket. For example, the manipulatable element 110 of an electrosurgical knife may be an electrical connection wire. For example, the manipulatable element 110 of a lithotripter may be an actuator shaft for reducing stones. For example, the manipulatable element 110 of an intraluminal balloon may be a lumen to secure and inflate and/or deflate a balloon. A coupling element 114 on the second end 106 may reversibly couple to the endoluminal accessory 112 (e.g., to the outer sheath 119 of the endoluminal accessory 112 or another component thereof). In some embodiments, an inner diameter of the outer sheath 119 (e.g., sheath 646 of FIG. 9A) of the endoluminal accessory 112 is sized to have a predefined interference with or friction on an outer diameter or surface of the manipulatable element 110 or another component (e.g., optional linkage 654, cable 656, or cable friction features 658 of FIG. 9A) to reduce or eliminate unintentional movement of the manipulatable element 110 in a lumen (e.g., lumen 647 of FIG. 9A) of the outer sheath 119. In some embodiments, at least a portion of the outer sheath 119 and the manipulatable element 110 within it may be considered the working length of the endoluminal accessory 112.

A clamping portion 116, in some embodiments, may slide along the shaft 102, for example within the groove 108, and reversibly clamp the manipulatable element 110 of the endoluminal accessory 112. When the clamping portion 116 is in a clamped state (i.e., as opposed to an unclamped state), the clamping portion 116 may manipulate the manipulatable element 110. In some embodiments, the clamped state of the clamping portion 116 permits the advancement and retraction of the manipulatable element 110 by the clamping portion 116 when the clamping portion 116 slides along the shaft 102. For example, in the embodiment shown in FIG. 2A, the clamping portion 116 is shown positioned in a first position near the first end 104 of the shaft 102. In the embodiment of FIG. 2B, the clamping portion 116 is instead shown positioned at a second position near the second end 106 of the shaft 102. Although a first position and a second position are shown in FIGS. 2A-2B, respectively, one of skill in the art will appreciate that any number of intermediate positions are possible and contemplated herein. For example, the clamping portion 116 may be positioned at any point along the length L2 of the shaft 102. As shown in the examples of FIGS. 2A-2B, the manipulatable element 110 has been repositioned accordingly with the position of the clamping portion 116. In some embodiments, the clamping portion 116 includes one or more interface features 117 (e.g., finger loops, partial loops, buttons, triggers, dials, wheels, etc.) to assist in the grasping or manipulation of the clamping portion 116 by a user.

For example, the manipulatable element 110 may include a wire such that sliding the clamping portion 116 distally (e.g., towards the second end 106 as shown in FIG. 2B) enlarges a loop in the wire (e.g., a snare of the endoluminal accessory 112) and sliding the clamping portion 116 proximally (e.g., towards the first end 104 as shown in FIG. 2A) reduces the loop in the wire (e.g., a snare of the endoluminal accessory 112). In some embodiments, a sliding of the clamping portion 116 that manipulates the manipulatable element 110 may open or close or otherwise actuate a grasper, a pair of forceps, a clip applier, a loop ligation accessory (e.g., PolyLoop®), an injection needle, a retrieval accessory, a retrieval basket, an electrosurgical knife, a sphincterotome, an extraction balloon, a lithotripter, intraluminal balloon, or a stent. In some embodiments, a sliding of the clamping portion 116 that manipulates the manipulatable element 110 may operate an injection needle. In some embodiments, a sliding of the clamping portion 116 that manipulates the manipulatable element 110 may expand or contract a basket, stent, balloon, etc. In some embodiments, manipulation of the manipulatable element 110 may activate or deactivate an electrosurgical knife, a sphincterotome, a lithotripter, etc.

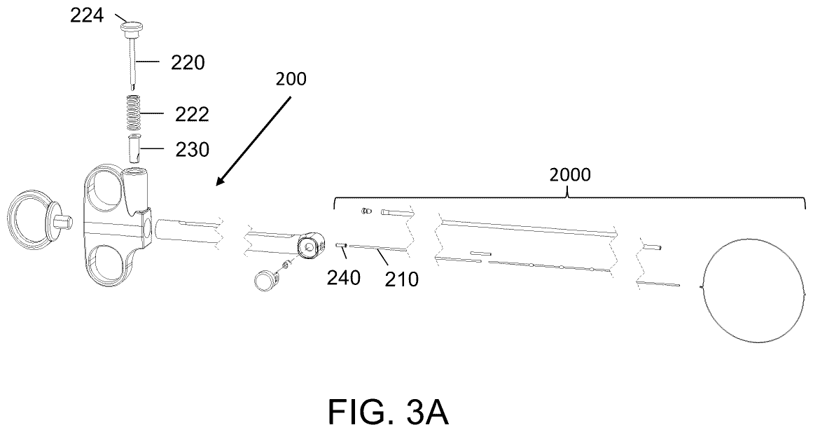

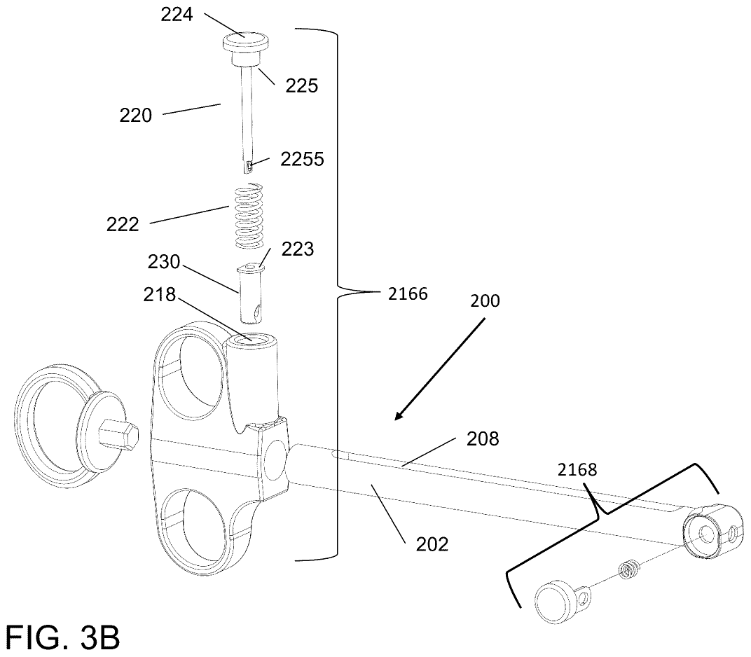

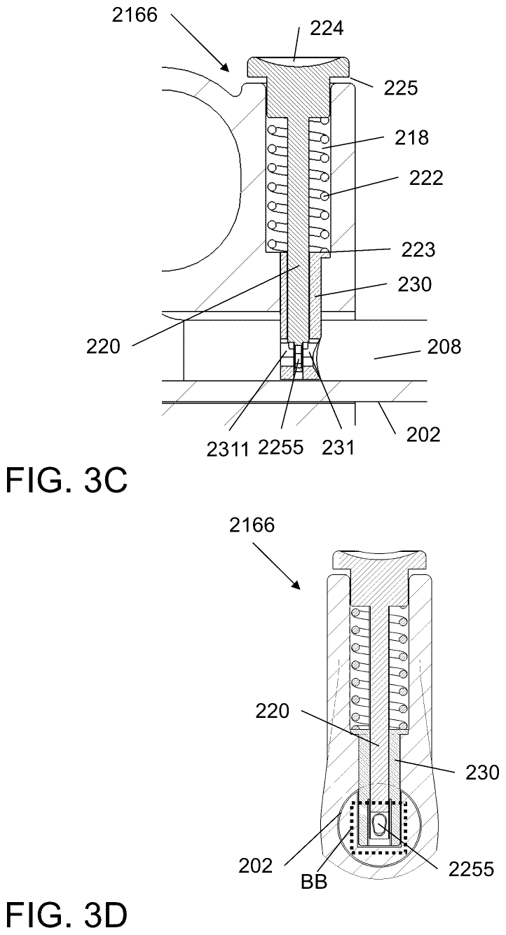

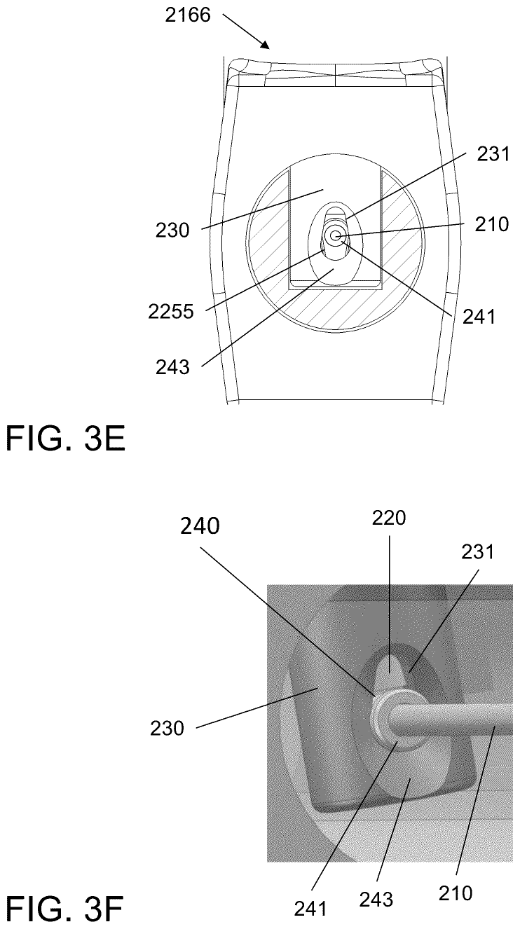

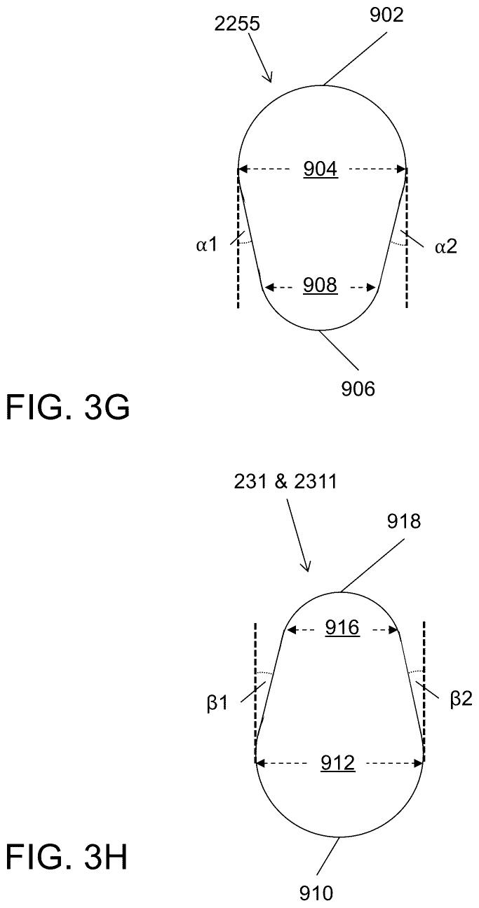

In some embodiments, the clamping portion 116 defines a channel 118 that may receive an elongate body 120. The elongate body 120 may clamp or unclamp the manipulatable element 110. In some embodiments, such as the embodiment shown in FIG. 2B, the elongate body 120 may include a screw, threaded structure, or push button to engage a corresponding feature of the channel 118. In some embodiments, the elongate body 120 may include a spring-loaded button, as shown in FIGS. 3A-3D.

FIG. 3A shows a clamping portion of a removable handle for engaging and retaining at least a portion of a snare. An example removable handle 200 is paired with a cable-operated snare-type accessory 2000 (e.g., an endoluminal accessory 112 of FIG. 2A).

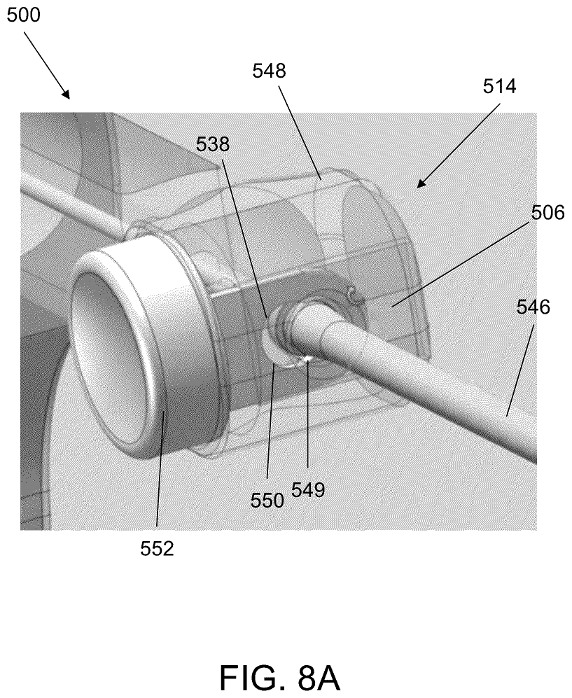

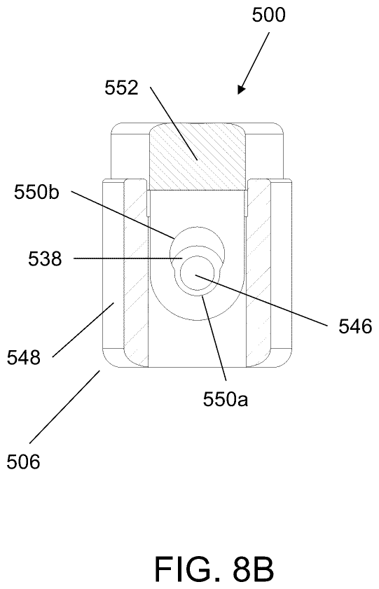

FIG. 3B illustrates the clamping portion of FIG. 3A having a channel for receiving a guide structure 230 and a coupling element disposed at an end of the shaft of a removable handle 200. The sliding component functions as the clamping portion, while the front-end rotatable lock or pushbutton lock, which secures the sheath of the endoluminal accessory, serves as a coupling element. An embodiment of a coupling element 2168 is shown in FIGS. 8A-8B.

Clamping portion 2166 defines a channel 218 that receives an elongate body 220 which may take the form of a spring-loaded button, as shown in FIG. 3B. The bottom of elongate body 220 has a clamping eyelet 2255 with a narrow portion and a wider portion (see, e.g., FIG. 3G). A resilient member 222 including, but not limited to, a compression spring may at least partially occupy the channel 218 and abut against an inner wall 223 of the guide structure 230 and the button bottom 225 of the elongate body 220. When the button top 224 of the elongate body 220 is depressed, the button bottom 225 compresses the resilient member 222 as the elongate body 220 moves from a first position towards a second position, sending the clamping eyelet 2255 deeper into the guide structure 230. In some embodiments, the first position is the clamped state, and the second position is the unclamped state. The compression of the resilient member 222 generates sufficient force to return elongate body 220 to the first position after the button top 224 is released. If the clamping eyelet 2255 and guide structure 230 are empty, elongate body 220 may instead move to a third position above the first position. Resilient member 222 remains compressed from the relaxed state even when not clamping anything, creating a preload that enables clamping orifices such as, the exit aperture 2311 and the entry aperture 231 (see, e.g., FIG. 3C) to apply a strong clamping force when in their first position.

FIG. 3C illustrates the guide structure 230 of FIG. 3B with a clamping eyelet 2255. In some embodiments, the elongate body 220 may include a clamping eyelet 2255 housed within a guide structure 230 that engages the proximal tip 240 of the manipulatable element 210 (as shown in FIG. 3F). For example, the clamping eyelet 2255 may be disposed between an entry aperture 231 and exit aperture 2311 of the guide structure 230. This configuration is implemented to clamp the proximal tip 240 in between the two tapered openings such as the exit aperture 2311 and the entry aperture 231 due to the opposing wedge shapes. In this example, the two wedges are about 15 degrees to about 25 degrees (internal angle in FIGS. 3G-3H), such that the tip is clamped by these opposing wedges. FIG. 3D illustrates the clamping eyelet 2255 of FIG. 3C positioned within an aperture of the shaft 202.

As shown in FIG. 3G, the clamping eyelet 2255, may have a tapered shape between a wider end 902 having a first diameter 904 and a narrower end 906 having a second diameter 908, such that the first diameter 904 is greater than the second diameter 908. As shown in FIG. 3H, the entry aperture 231 and exit aperture 2311 may have a tapered shape between a wider end 910 having a third diameter 912 and a narrower end 918 having a fourth diameter 916, of which the third diameter 912 is greater than the fourth diameter 916. Angle α1, angle α2, angle β1, and angle β2, which define a slope transitioning from the first diameter 904 to the second diameter 908 and the third diameter 912 to the fourth diameter 916, respectively as shown, may be about 2 degrees to about 35 degrees, about 15 degrees to about 35 degrees, about 10 degrees to about 25 degrees, about 12 degrees to about 30 degrees, about 5 degrees to about 30 degrees, about 8 degrees to about 20 degrees, about 8 degrees to about 15degrees, about 5 degrees to about 15 degrees, about 8 degrees to about 12 degrees, or about 10 degrees in some embodiments. For example, the eyelet or aperture shown in FIGS. 3G-3H has an angle of about 20 degrees, where alpha and beta each measure about 10 degrees. In some embodiments, angles α1, angle α2, angle β1, and angle β2 may have the same value. In some embodiments, at least two of angle α1, angle α2, angle β1, and angle β2 may have the same value. In some embodiments, angle α1, angle α2, angle β1, and angle β2 are each different.

In some embodiments, the clamping eyelet 2255 is arranged with entry aperture 231 and exit aperture 2311 such that an edge of the wider end 910 of the entry aperture 231 and exit aperture 2311 are aligned with an edge of the narrower end 906 of the clamping eyelet 2255 and an edge of the narrower end 918 of the entry aperture 231 and exit aperture 2311 are aligned with an edge of the wider end 902 of the clamping eyelet 2255 (i.e., when placed side-by-side, the wider end 902 of the clamping eyelet 2255 is opposite the narrower end 918 of both the entry aperture 231 and the exit aperture 2311).

FIGS. 3E-3F show the clamping eyelet 2255 of FIG. 3D partially retaining the manipulatable element 210. In other words, at least a portion of the manipulatable element 210 is grasped and retained by the clamping eyelet 2255. In reference to FIG. 3E, which shows a detailed view of Box BB of FIGS. 3D-FIG. 3F, the manipulatable element 210 may include or terminate with the proximal tip 240 which may include a flange 241 that has dimensions wider than a diameter of the proximal tip 240. In some embodiments, the proximal tip 240 fits tightly or is too wide to pass through the narrower end 906 of the clamping eyelet 2255 or the narrower end 918 of the entry aperture 231 or the exit aperture 2311 but narrow enough to fit through the wider end 910 of the entry aperture 231 and the exit aperture 2311. In some embodiments, the entry aperture 231 also has a countersink to facilitate insertion of proximal tip 240. Further, in an example embodiment, the orifice walls form an angle of approximately 20 degrees or within a range of about 5 degrees to about 45 degrees. The configuration is implemented to exert force using a wedge-like shape. If the wedge angle is too large (e.g., approximately 60 degrees), a greater spring force would be required to clamp the proximal tip 240. Conversely, if the wedge angle is too small (e.g., approximately one degree), the elongate body 220 may translate a greater distance to open the orifice to receive the proximal tip 240. The dimensions of the flange 241, in some embodiments, prevent the flange 241 from passing through either the wider end 910 or the narrower end 918 of the entry aperture.

While in the clamped state, in some embodiments, the resilient member 222 can exert a force on the elongate body 220 such that clamping eyelet 2255 lifts the proximal tip 240 up into a first position to trap the proximal tip 240 using friction against sidewalls of the narrower end 906 of the clamping eyelet 2255 and the narrower end 918 of the entry aperture 231 and exit aperture 2311. While in the unclamped state, in some embodiments, the button top 224 is depressed to compress the resilient member 222 and move the elongate body 220 and therefore the clamping eyelet 2255 to a second position such that the proximal tip 240 is loose in the wider end 902 of the clamping eyelet 2255 and the wider end 910 of the entry aperture 231 and exit aperture 2311, allowing the manipulatable element 210 to be removed from the clamping eyelet 2255, entry aperture 231, and exit aperture 2311. The flange 241 may prevent the proximal tip 240 from fully passing through the entry aperture 231, clamping eyelet 2255, and exit aperture 2311. The flange 241 may provide a reliable, specific position of attachment for the clamping portion 2166 on the manipulatable element 210, in some embodiments, when the proximal tip 240 is positioned with the flange 241 abutting against the guide structure 230. In some embodiments, the guide structure 230 may include a countersink 243 to deflect the proximal tip 240 into the entry aperture 231. When in a clamped state, the clamping portion 2166 may move the manipulatable element 210 within the groove 208 of the shaft 202.

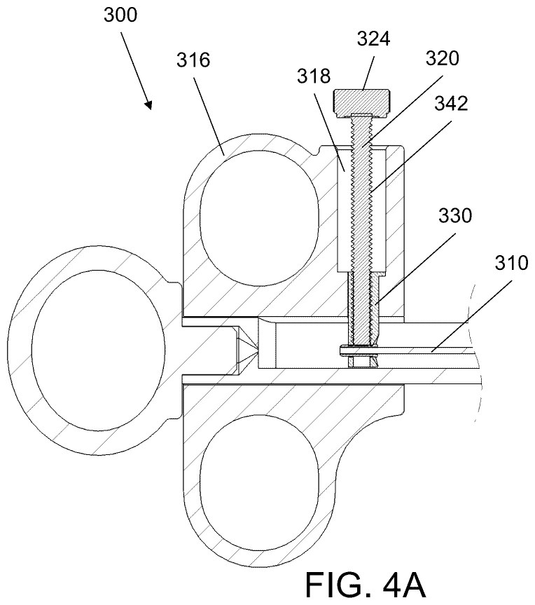

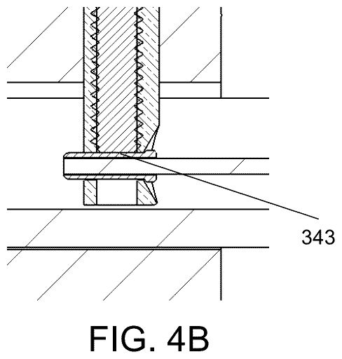

FIGS. 4A-4B show a partial, zoomed-in, cross-sectional view of an embodiment of a removable handle 300. The removable handle 300 may include a clamping portion 316 defining a channel 318 with an elongate body 320 including a threaded structure 342. In some embodiments, a guide structure 330 includes a corresponding structure to receive the threaded structure 342. In some embodiments, the clamping portion 316 includes a clamping end 343 of the elongate body 320. While in a clamped state, in some embodiments, the threaded structure 342 of the elongate body 320 may be tightened using knob 324 to a first position such that the clamping end 343 pinches an inserted manipulatable element 310 (e.g., with or without the proximal tip 240 of FIGS. 3A-3B) against the guide structure 330. While in an unclamped state, in some embodiments, the elongate body 320 may be loosened by the threaded structure 342 to a second position such that the clamping end 343 provides insufficient friction to hold the manipulatable element 310 within the guide structure 330. In some embodiments, the elongate body 320 may further include an electrode or electrical contact (not shown) that may be used to apply a current to a portion of the manipulatable element 310, thereby powering a portion of the endoluminal accessory (e.g., endoluminal accessory 112 of FIGS. 2A-2B, endoluminal accessory 612 of FIG. 9A). For example, the electrode or electrical contact may empower a lithotripsy accessory (e.g., a laser, mechanical, or ultrasonic lithotripsy accessory). For example, the electrode or electrical contact may empower an electrosurgical knife.

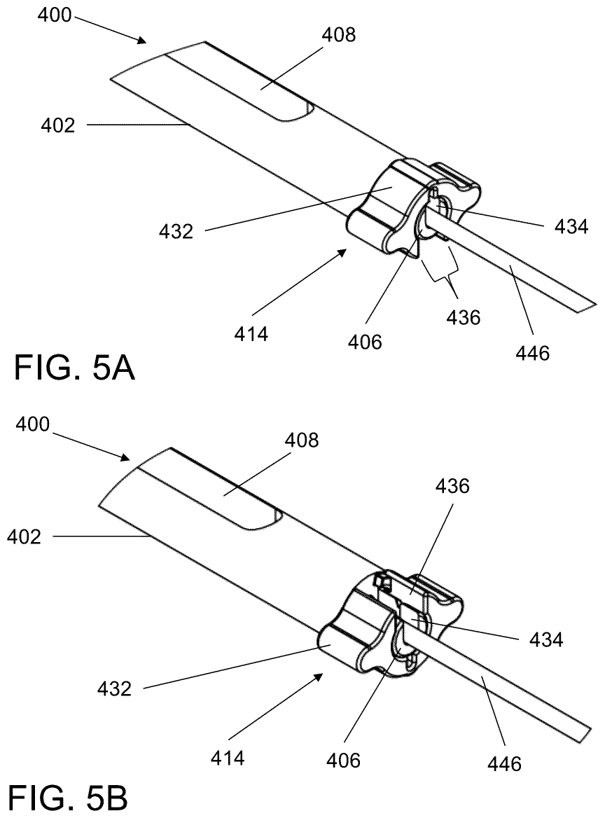

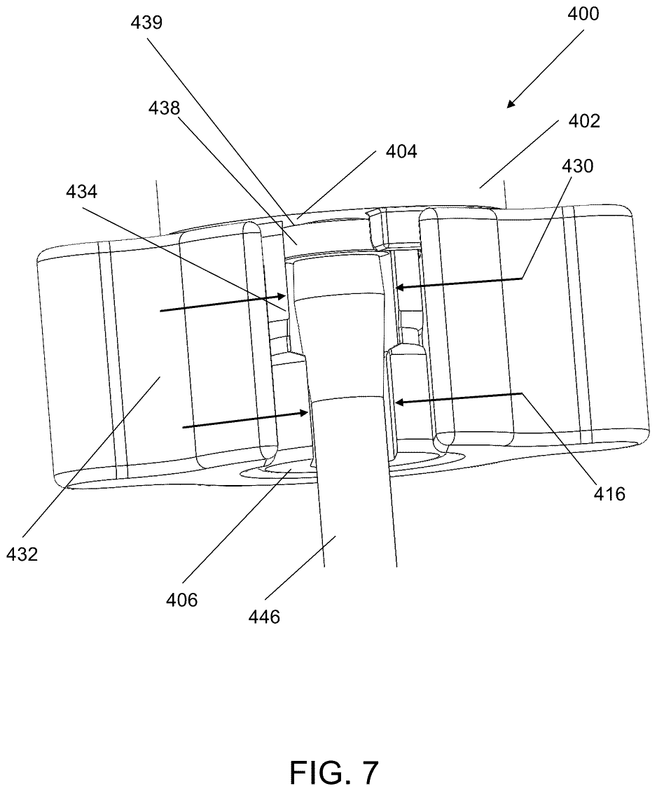

FIGS. 5A-5B depict a first and second position, respectively, of another embodiment of a coupling element 414 located on a second end 406 of a removable handle 400. In some embodiments, the coupling element 414 includes a rotatable lock 432 that may rotate about a channel 434 of the second end 406 of the shaft 402 (e.g., of the removable handle 400). To retain at least a portion of the sheath 446. The channel 434, in some embodiments, may have a U-shape, V-shape, or another profile, and may receive a proximal end of an endoluminal accessory (e.g., endoluminal accessory 112 of FIGS. 2A-2B, endoluminal accessory 612 of FIG. 9A). In some embodiments, the proximal end may be a manipulatable element (e.g., manipulatable element 110 of FIGS. 2A-2B, manipulatable element 210 of FIG. 3B, etc.) or an extension thereof which at least partially extends through the channel 434 into the groove 408 of the shaft 402. In other words, the channel 434 is sized and/or shaped to receive a proximal end, such as sheath 446 which may enclose a manipulatable element, and the associated flange 438 (see, e.g., FIG. 7) of an endoluminal device accessory (e.g., endoluminal device accessory 112 of FIGS. 2A-2B). In an embodiment, the sheath 446 may enclose a manipulatable element (not shown), (e.g., the manipulatable element 110 of FIGS. 2A-2B, the manipulatable element 210 of FIG. 3B), or an extension thereof. The manipulatable element extends out through the proximal end of the sheath 446, passing through the flange 438 and into the groove 408 of the shaft 402. The proximal end of the sheath 446 and the flange 438 are positioned within the channel 434 and at least partially extend into the groove 408 of the shaft 402. Said another way, the proximal surface of flange 438 of flanged guide 439 abuts against surface 404, as shown in FIG. 7.

The rotatable lock 432 may rotate around the sheath 446 and flange 438 for securing in a first position (e.g., a locked state) or releasing in a second position (e.g., an unlocked state) the sheath 446 of the endoluminal accessory (e.g., endoluminal accessory 112 of FIGS. 2A-2B, endoluminal accessory 612 of FIG. 9A). Although the first position and second position are shown at about 180-degree positions, one of skill in the art will appreciate, and it is contemplated herein, that the first and second positions may be at any circumferential position. For example, the first position may be between about 0 degrees and about 180 degrees, about 10 degrees and about 170 degrees, about 20 degrees and about 160 degrees, about 30 degrees and about 150 degrees, about 60 degrees and about 120 degrees, about 0 degrees and about 90 degrees, and about 90 degrees and about 180 degrees. The second position may be between about 180 degrees and about 360 degrees, about 190 degrees and 350 degrees, about 200 degrees and 340 degrees, about 210 degrees and 330 degrees, about 240 degrees about 300 degrees, about 180 degrees and 270 degrees, and about 270 degrees, and 360 degrees.

In some embodiments, the rotatable lock 432 defines a lock channel 436. When the lock channel 436 is positioned such that the open portion aligns with the channel 434 of the shaft 402 (e.g., in the second position), the rotatable lock 432 is in an unlocked position, allowing access to channel 434, as illustrated in FIG. 5B. In an example implementation, when the rotatable lock 432 is rotated approximately 180 degrees, the bottom surface of the lock channel 436 obstructs the open end of channel 434, thereby preventing the insertion or removal of a component through channel 434. In some embodiments, when the rotatable lock 432 is in the unlocked position, the removable handle 400 may be decoupled from the endoluminal accessory (e.g., endoluminal accessory 112 of FIGS. 2A-2B, endoluminal accessory 612 of FIG. 9A). In an example, the proximal end or sheath 446 is lifted to induce flexion in the sheath 446 and the inner manipulatable element, thereby elevating the flanged guide 439 above and clear of a narrow end 416 (FIG. 7) of the channel 434. Once in this position, the sheath 446, along with the flanged guide 439 and the inner manipulatable element, can be withdrawn from channels 434 and 436, and subsequently from shaft 402. The rotatable lock 432 functions to restrict upward movement of the sheath 446 and the flanged guide 439, thereby preventing disengagement from channel 434 and ensuring that the components remain securely connected. As noted above, channel 434 has a primary capturing structure which is a narrow end 416 of the channel 434 for retaining a first portion of the sheath 446 and a secondary capturing structure which is a wider end 430 for retaining a second portion of the sheath 446. The first portion of the sheath 446 has a first diameter that is different than a second diameter of the second portion of the sheath 446.

When the lock channel 436 is offset from, or rotationally misaligned with (e.g., by about 15 degrees to about 180 degrees, about 15 degrees to about 150 degrees, about 15 degrees to about 120 degrees, about 15 degrees to about 90 degrees, etc.), the channel 434 of the shaft 402, (e.g., in the first position) such that the open portion of the channel 434 is blocked by the rotatable lock 432, the rotatable lock 432 is in a locked position, in some embodiments, as shown in FIG. 5A. When the rotatable lock 432 is in a locked position, a proximal end of a sheath of an endoluminal accessory (e.g., endoluminal accessory 112 of FIGS. 2A-2B, endoluminal accessory 612 of FIG. 9A) may not be inserted into or removed from the channel 434 of the shaft 402, in some embodiments.

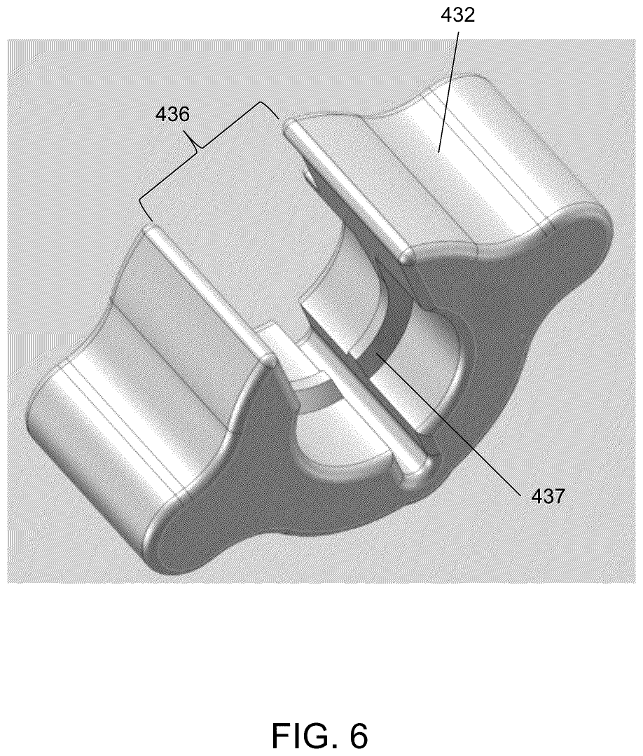

FIG. 6 shows a perspective view of an embodiment of a portion of a rotatable lock 432 separate from a removable handle. In some embodiments, the rotatable lock 432 defines a lock channel 436 having a reduced diameter section 437. In some embodiments, the reduced diameter section 437 may be formed by a ridge or lip passing through at least a portion of the lock channel 436.

FIG. 7 shows a rotatable lock 432 in an unlocked position about a second end 406 of an embodiment of a removable handle 400. In some embodiments, an endoluminal accessory (e.g., endoluminal accessory 112 of FIGS. 2A-2B, endoluminal accessory 612 of FIG. 9A) may include a sheath 446 that defines a lumen (e.g., lumen 647 of FIG. 9A) within which the proximal end of the manipulatable element (e.g., manipulatable element 110 of FIGS. 2A-2B, manipulatable element 210 of FIG. 3A, etc.), or an extension thereof, may pass. The sheath 446, in some embodiments, may include or may be coupled to a flange guide 439 that has a flange 438 which extends beyond the diameter of the sheath 446. The flange 438 may be positioned on a surface 404 of the shaft 402 of the removable handle 400. In some embodiments, the flange 438 has a diameter greater than that of the lumen 609 of FIG. 9A, preventing the entry into device lumen 609 due to the obstruction provided by surface 404.

When the rotatable lock 432 is in the locked position, the reduced diameter section 437 engages with the flange 438 within the channel 434 of the shaft 402 and is preventing the proximal end of the endoluminal accessory (e.g., endoluminal accessory 112 of FIGS. 2A-2B, endoluminal accessory 612 of FIG. 9A) from decoupling from the removable handle 400. Flange 438 has a diameter that is larger than the lumen (e.g., lumen 609 of FIG. 9A) of the distal end (e.g., second end 106) of the removable handle 400, in some embodiments, which therefore prevents the sheath 446 from entering the groove 408 of the removable handle 400. When the rotatable lock 432 is in the unlocked position, the reduced diameter section 437 is disconnected from the channel 434 of the shaft 402 and the flange 438 of the flange guide 439 such that the endoluminal accessory may be withdrawn from the channel 434. In at least this manner, the reduced diameter section 437 may be considered as a stop about at least a portion of the flange 438.

FIG. 8A depicts an embodiment of a coupling element 514 of a removable handle 500, and FIG. 8B depicts a partial cross section of an embodiment of a coupling element 514. In some embodiments, the coupling element 514 includes a housing 548 with a coupling hole 549 capable of receiving a sheath 546 fitted with a flanged guide (e.g., flanged guide 639 of FIG. 9A) having a flange 538. The coupling element 514, in some embodiments, may also define a variable diameter eyelet 550 having a first diameter portion 550a (or reduced diameter portion) and a second diameter portion 550b (or enlarged diameter portion). The first diameter portion 550a may have a diameter that is less than, or reduced as compared to, the diameter of the second diameter portion 550b. The coupling element 514, in some embodiments, may also include an input element 552. In some embodiments, the flange 538 of the flanged guide (e.g., flanged guide 639 of FIGS. 9A-9B) has a diameter that is greater than the diameter of the reduced diameter portion 550a of the variable diameter eyelet 550 (such that it cannot pass through the reduced diameter portion 550a) but may pass through the second diameter portion, having a diameter greater than the reduced diameter portion. The smaller diameter of the lumen 609 prevents flanged guide 639 with its flange 638 from entering the channel 608, as is shown in FIGS. 9A-9B.

In some embodiments, a deactivation of the input element 552 (e.g., spring-loaded button) causes interaction between the reduced diameter portion 550a of the variable diameter eyelet 550 with a flange 538 of the flanged guide (e.g., flanged guide 639 of FIGS. 9A-9B) to lock the endoluminal accessory (e.g., endoluminal accessory 112 of FIGS. 2A-2B, endoluminal accessory 612 of FIG. 9A) in the second end 506 of the removable handle 500. Activation of the input element 552 causes interaction between an enlarged diameter portion 550b of the variable diameter eyelet 550 with the flange 538 of the flanged guide (e.g., flanged guide 639 of FIGS. 9A-9B) to unlock the endoluminal accessory (e.g., endoluminal accessory 112 of FIGS. 2A-2B, endoluminal accessory 612 of FIG. 9A) in the second end 506 of the removable handle 500. For example, when the input element 552 is activated, the enlarged diameter portion 550b of the variable diameter eyelet 550 may align with the coupling hole 549 of the housing 548, allowing the flange 538 of the flanged guide (e.g., flanged guide 639 of FIGS. 9A-9B) to enter or be removed from the housing 548. When the input element 552 is deactivated, the reduced diameter portion 550a of the variable diameter eyelet may align with the coupling hole 549 of the housing 548, trapping the flange 538 of the flanged guide (e.g., flanged guide 639 of FIGS. 9A-9B) within the housing 548.

FIG. 9A shows a cross-section of an exploded view of an embodiment of a removable handle 600 for an endoluminal accessory 612. FIG. 9B shows a zoomed in, cross-sectional view of a distal portion of the removable handle 600 of FIG. 9A. The removable handle 600 includes a shaft 602 having a first end 604 (proximal end) and a second end 606 (distal end) and defining a groove 608, which is an opening bordering the lumen of the shaft 602. A clamping portion 616 defines a channel 618 that may receive an elongate body 620. In this embodiment, the elongate body 620 includes a threaded structure that engages a corresponding threaded feature of a guide structure 630. In some embodiments, the clamping portion 616 reversibly clamps to a clamping tip 640 of a manipulatable element 610 of an endoluminal accessory 612. In some embodiments, an optional linkage 654 may connect the manipulatable element 610 to a cable 656 of the endoluminal accessory 612. In some embodiments, no linkage 654 is present as the manipulatable element 610 is an extension of the cable 656. In some embodiments, the manipulatable element 610 may be made of nitinol or another material that is medically safe, capable of transferring force from a motion of the clamping portion 616 without kinking or breaking, and/or flexible enough to be removed from the removable handle 600 without damage. For example, nitinol wire may provide column strength to transmit push and pull forces to the cable for operating a terminal device. At the same time, it remains flexible enough to bend off-axis during insertion and removal of the cable assembly without permanently bending and/or kinking. A permanent bend and/or kink may reduce the efficiency of force transmission.

The manipulatable element 610, optional linkage 654, and cable 656 may at least partially occupy a lumen 647 of a sheath 646 of the endoluminal accessory 612. In some embodiments, the lumen 647 of the endoluminal accessory 612 may include one or more lumen friction features (e.g., bumps, ridges, spikes, hairs, springs, bent nitinol wires, coils, etc.) that reduce passive movement of the manipulatable element 610 or cable 656 in the lumen 647 of the endoluminal accessory 612. In some embodiments, the manipulatable element 610, optional linkage 654, and/or cable 656 may include cable friction features 658 (e.g., bumps, nubs, spikes, nodes having a wider diameter, threaded features, silicone tubing with interference fit, etc.) that reduce or prevent passive or unintended movement of the manipulatable element 610 or cable 656 in the lumen 647 of the endoluminal accessory 612. In some embodiments, cable friction features 658 may engage with lumen friction features. Lumen friction features and/or cable friction features 658 may help to reduce undesired movement of the manipulatable element 610 and/or cable 656 while the removable handle 600 is detached from the endoluminal accessory 612. In some embodiments, at least a portion of the sheath 646 and the portions of the manipulatable element 610, optional linkage 654, and cable 656 within the sheath 646 may be considered the working length of the endoluminal accessory 612.

A flanged guide 639 may connect to the sheath 646 and assist in the connecting of the endoluminal accessory 612 (e.g., including the sheath 646, manipulatable element 610, optional linkage 654, and cable 656) to the removable handle 600, in some embodiments. A coupling element 614 may secure the endoluminal accessory 612 to the removable handle 600. In some embodiments, the coupling element 614 may interact with the flanged guide 639 to secure the endoluminal accessory 612 to the removable handle 600.

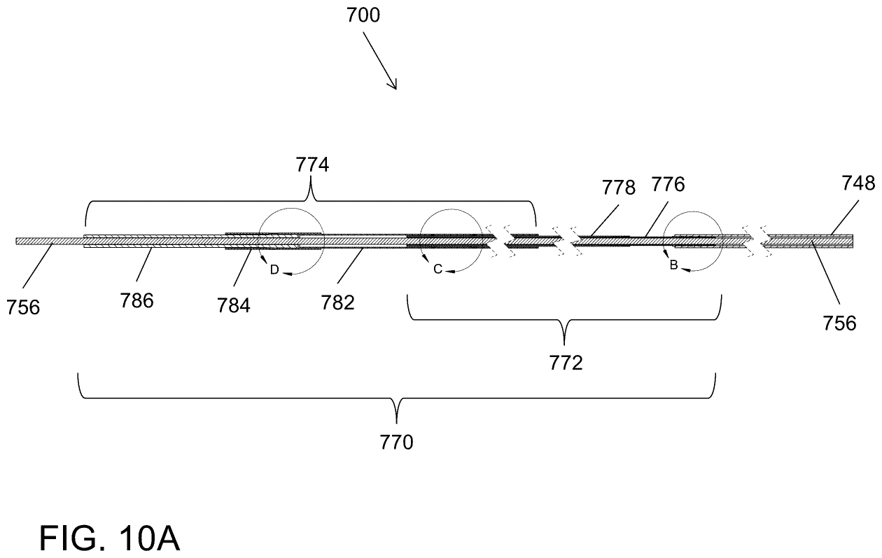

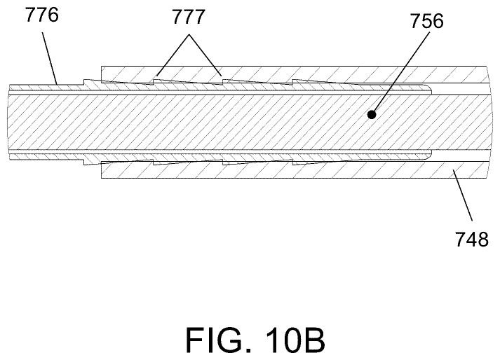

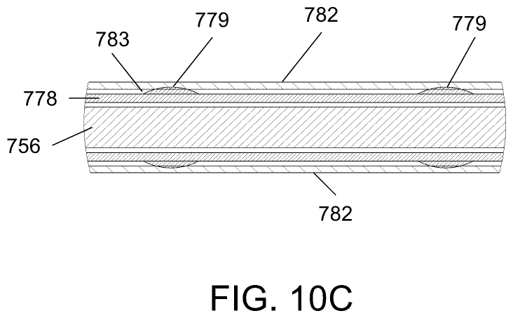

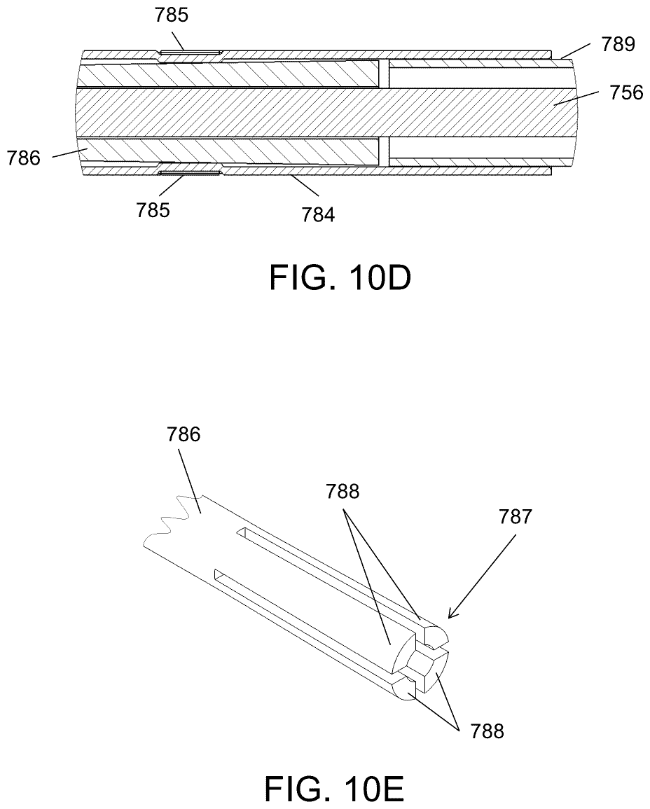

FIG. 10A shows a system 700 including a removable handle 770 for removable attachment to an endoluminal accessory (e.g., sheath 748). The removable handle 770 may have a low-profile and/or be working channel compatible. In some embodiments, the removable handle 770 is couplable to a sheath 748 retaining a cable 756 (e.g., the sheath 748 and cable 756 forming part of the endoluminal accessory).

FIG. 10B shows the sheath 748 of detail B of FIG. 10A removably coupled with an adapter 776. The adapter 776 includes one or more attachment elements 777 (e.g., barbs, anchors, friction bumps, etc.) for securing the adapter 776 in the sheath 748.

FIG. 10C illustrates the outer friction sleeve 782 of detail C of FIG. 10A with an example implementation of a friction length 778. Outer friction sleeve 782 is shown in FIG. 10C with no friction receivers 783.

FIG. 10D illustrates the sheath 748 of detail D of FIG. 10A with an example of a cable grip 786 to be inserted around the cable 756 and into a crimp hypotube 784 having one or more crimps 785.

FIG. 10E illustrates the cable grip 786 of FIG. 10D with a distal end having one or more fingers 788.

Now referring to FIG. 10A, in some embodiments, the system 700 may include a handle 770 including an adapter 776 for modifying an endoluminal accessory (e.g., endoluminal accessory 112 of FIGS. 2A-2B, endoluminal accessory 612 of FIG. 9A) having a sheath 748 and a cable 756. For example, the endoluminal accessory (e.g., endoluminal accessory 112 of FIGS. 2A-2B, endoluminal accessory 612 of FIG. 9A) may be an off-the-shelf accessory with the original handle irreversibly removed. In some embodiments, a portion of the sheath 748 may be removed to expose a length of cable 756 to mate with the handle 770.

The handle 770 may include, in some embodiments, a stabilizing assembly 772 and a mobile assembly 774. The stabilizing assembly may include an adapter 776 (e.g., sheath-coupling hypotube) coupled to a friction length 778, in some embodiments. The adapter 776, made of, for example, medical-grade stainless steel, in some embodiments, may include one or more attachment elements 777, such as ridges or barbs, on at least a portion of the distal end. The one or more attachment elements 777, when inserted between the cable 756 and sheath 748, secure the adapter 776 to the sheath 748 while allowing the cable 756 to pass within the adapter 776, as shown in FIG. 10B, which depicts detail B of FIG. 10A in greater detail. A friction length 778, for example, comprising polyimide, another plastic, or a combination thereof, in some embodiments, may be affixed to the external diameter of the adapter 776. In some embodiments, the friction length 778 may include adapter friction features 779 (e.g., bumps, ridges, spikes, hairs, springs, bent nitinol wires, coils, etc.) on the external surface to engage one or more friction receivers 783 (e.g., valleys, holes, gaps, grooves, etc.) of an outer friction sleeve 782 of the mobile assembly 774 as seen in FIG. 10C, which depicts detail C of FIG. 10A in greater detail. In some embodiments, the friction length 778 has no friction receivers 783, and the adapter friction features 779 instead engage the inner diameter of the outer friction sleeve 782. The interaction between the adapter friction features 779 of the friction length 778 with the friction receivers 783 of the outer friction sleeve 782 or the inner diameter of the outer friction sleeve 782 may help prevent or reduce unwanted sliding of the cable 756 when the cable 756 is disconnected from the endoluminal accessory (e.g., endoluminal accessory 112 of FIGS. 2A-2B, endoluminal accessory 612 of FIG. 9A), in some embodiments.

As noted above, in some implementations, a friction receiver 783 may not be present. Instead, the adapter friction features 779 having an outer diameter (OD) larger than the inner diameter (ID) of the mating tube (e.g., outer friction sleeve 782) may be present to create a light interference fit. Upon insertion, the adapter friction features 779 elastically or temporarily deform the tubing in the areas of contact, ensuring engagement to prevent movement of the mobile assembly 774 (and thereby cable 756) without permanent deformation. Additionally, an internal friction receiver zone may be considered as an alternative implementation. This configuration is applicable to the previously described embodiments including, but not limited to, the shaft for handle 200, handle 300, handle 400, and/or handle 500.

In some embodiments, the mobile assembly 774 may feature an outer friction sleeve 782 affixed to a crimp hypotube 784 with the crimp hypotube 784 further coupled to a cable grip 786. In some embodiments, the grip length 789 may be made of polyimide or another plastic and is bonded to the crimp hypotube 784, which may be made of medical grade stainless steel. The cable grip 786 may be made of, for example, medical grade stainless steel, in some embodiments, and may feature at a distal end 787 one or more fingers 788, like those of a collet chuck, which taper to have a greater thickness at the distal end 787, as shown in FIG. 10E. In some embodiments, the cable grip 786 has four fingers 788. In some embodiments, the cable grip 786 may have two fingers 788, or three fingers 788, or some number of fingers 788 greater than four.

The distal end 787 of the cable grip 786 is inserted into the crimp hypotube 784 with cable 756 passing through it, in some embodiments. In addition to a tight or friction fit between the dimensions of the crimp hypotube 784 and the cable grip 786, the crimp hypotube 784 may include one or more crimps 785, as shown in FIG. 10D (which is depicted in detail D of FIG. 10A in more detail), that apply force onto the fingers 788 of the cable grip 786. With the cable grip 786 fitted within the crimp hypotube 784, the mobile assembly 774 may move with the cable 756 relative to the stabilizing assembly 772 and sheath 748 as much as is allowed by the interaction of outer friction sleeve 782 and the friction features (such as adapter friction features 779).

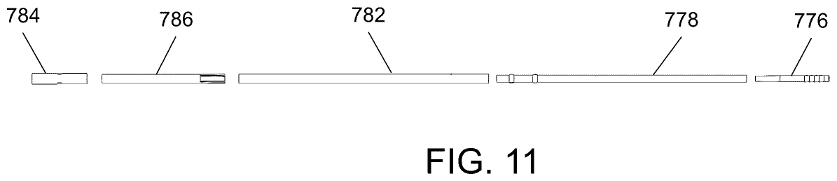

FIG. 11 illustrates the removable handle 770 of FIG. 10A with various components, as noted above, and self-retaining features that may or may not be coupled with the outer sheath 748 and inner cable 756 of the endoluminal accessory (e.g., endoluminal accessory 112 of FIGS. 2A-2B, endoluminal accessory 612 of FIG. 9A).

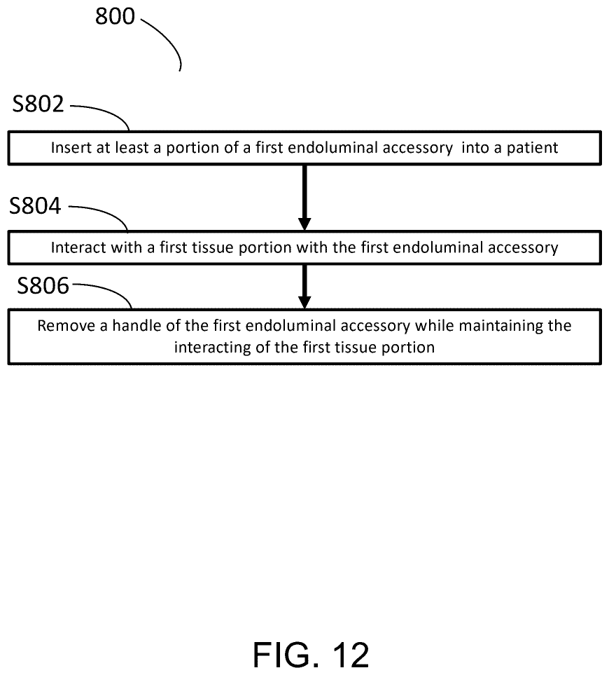

FIG. 12 shows an embodiment of a method 800 for removing a handle from an endoluminal accessory. In general, the method may include inserting at least a portion of a first endoluminal accessory into a patient. For example, at least a portion of the first endoluminal accessory may be inserted directly into a patient's anatomy or into a working channel of a first endoluminal instrument. The first endoluminal instrument may be an endoscope, echoendoscope, catheter, trocar, sheath, etc. The first endoluminal instrument may also be those that include or provide a “temporary working channel” such as Wilson-Cook's Sew-Right & Ti-Knot devices. The method may include interacting with a first tissue portion with the first endoluminal accessory. For example, interacting may include grasping, cauterizing, expanding, applying energy, etc. The method may include removing a handle of the first endoluminal accessory while maintaining interaction with the first tissue portion. The method functions to reversibly attach a handle to a first accessory compatible with a first endoluminal instrument having a working channel (e.g., endoscope, catheter, sheath, trocar, etc.). The method may be used for endoscopy procedures, thrombectomy procedures, ligation procedures, ablation procedures, ultrasound procedures, electrocautery procedures, etc.

In some embodiments, the method 800 may include inserting at least a portion of a first endoluminal accessory into a patient in block S802, interacting with a first tissue portion with the first endoluminal accessory in block S804, and removing a handle of the first endoluminal accessory while maintaining the grasping of the first tissue portion in block S806. In block S802, the method 800 includes inserting at least a portion of a first endoluminal accessory into a patient. In some embodiments, at least a portion of the first endoluminal accessory may be inserted directly into a patient's anatomy or into a working channel of a first endoluminal instrument. In some embodiments, the first endoluminal instrument may be an endoscope. Although an endoscope is described as the first endoluminal instrument with respect to method 800, any device with a working channel, “temporary” working channel, or lumen may be used for the first endoluminal instrument, for example, a catheter, trocar, sheath, etc. In an implementation, a temporary working channel may be affixed externally to an endoscope to facilitate instrument passage. For instance, some surgical fastening devices incorporate external auxiliary working channels to enable specific procedural applications. Additionally, the first endoluminal device may be employed independently, without the use of an endoscope. However, in some cases, the user may wish to remove the handle while keeping the device in place for procedural or operational reasons.

The first endoluminal accessory may be an accessory (e.g., endoluminal accessory 112 of FIGS. 2A-2B, endoluminal accessory 612 of FIG. 9A) with a removable handle as described herein. In some embodiments, the first endoluminal accessory may be an accessory fitted with a removable handle (e.g., handle 770 of FIG. 10A) as described herein. In some embodiments, the first endoluminal accessory may be, but is not limited to, a miniature through-the-channel scope, a snare, a grasper, a forceps, a clip applier, a loop ligation accessory, an injection needle, a retrieval accessory, a retrieval basket, an electrosurgical knife, a sphincterotome, an extraction balloon, or a lithotripter. In some embodiments, the first endoluminal accessory may be a snare.

In block S804, the method 800 may include interacting with a first tissue portion with the first endoluminal accessory, in some embodiments. In embodiments in which the first endoluminal accessory is a snare, the interacting with the first tissue portion may include grasping the first tissue portion such as by closing or tightening the snare. In embodiments in which the first endoluminal accessory is a scope, interacting with the first tissue portion may include repositioning the scope for a different view or illumination of the first tissue portion. In embodiments in which the first endoluminal accessory is a grasper, interacting with the first tissue portion may include a pinching or tightening of the grasper. In embodiments in which the first endoluminal accessory is a forceps, interacting with the first tissue portion may include a pinching or tightening of the forceps. In embodiments in which the first endoluminal accessory is an injection needle, interacting with the first tissue portion may include injecting at least a portion of a substance from the injection needle into the first tissue portion. In embodiments in which the first endoluminal accessory is an electrosurgical knife, interacting with the first tissue portion may include making an incision into the first tissue portion.

In block S806, the method 800 may include removing a handle of the first endoluminal accessory while maintaining the interaction with the first tissue portion. In some embodiments, the handle is a removable handle (e.g., removable handle 100 of FIGS. 2A-2B, etc.) as described herein. In some embodiments, the removing of the handle may include placing a clamping portion (e.g., clamping portion 116 of FIGS. 2A-2B) in an unclamped state. For example, placing a clamping portion into an unclamped state may include pressing or releasing a spring-loaded button (e.g., button top 224 of FIG. 3A) or loosening a threaded structure (e.g., threaded structure 342 of FIG. 4A). In some embodiments, the removing of the handle may include positioning a coupling element (e.g., coupling element 114 of FIGS. 2A-2B) in an unlocked position. For example, positioning a coupling element into an unlocked position may include rotating a rotatable lock (e.g., rotatable lock 432 of FIGS. 5A-5B) or pressing or releasing an input element (e.g., the spring-loaded button as input element 552 of FIG. 8A). In some embodiments, lumen friction features, cable friction features (e.g., cable friction features 658 of FIG. 9A), adapter friction features (e.g., adapter friction features 779 of FIG. 10C) and/or friction receivers (e.g., friction receivers 783 of FIG. 10C) may at least substantially hold a cable (e.g., cable 656 of FIG. 9A) or a manipulatable element (e.g., manipulatable element 110 of FIGS. 2A-2B) in place (i.e., maintain a position) while the handle is being or has been removed and therefore may maintain the interaction with (e.g., grasping of) the first tissue portion.

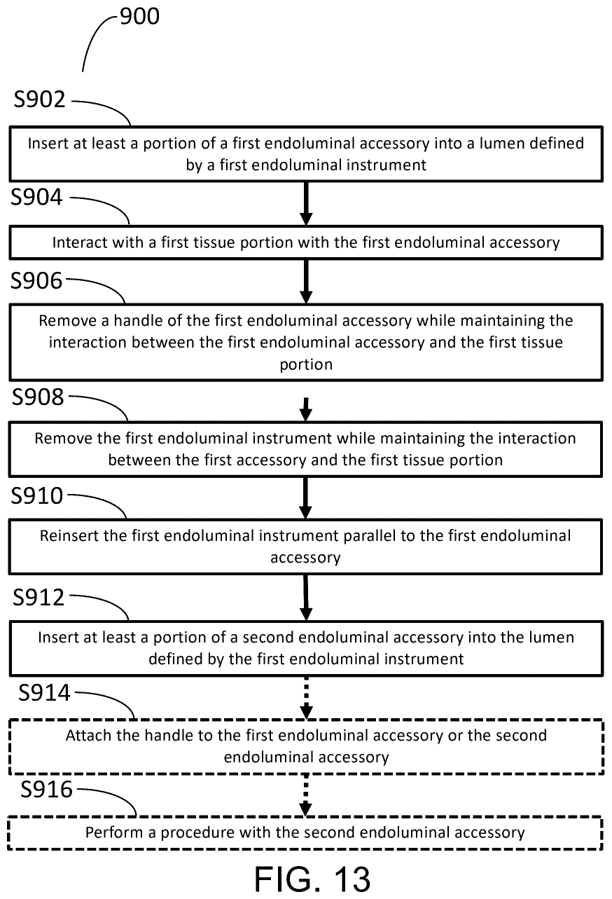

FIG. 13 shows an embodiment of a method 900, for accessing a bodily region with one or more endoluminal accessories. In general, the method may include inserting at least a portion of a first endoluminal accessory into a lumen defined by a first endoluminal instrument. For example, the at least a portion of the first endoluminal accessory may be inserted into a working channel of a first endoluminal instrument. The first endoluminal instrument may be an endoscope, echoendoscope, catheter, trocar, sheath, etc. The first endoluminal instrument may also be those that include or provide a “temporary working channel” such as Wilson-Cook's Sew-Right & Ti-Knot devices. The method may include interacting with a first tissue portion with the first endoluminal accessory. For example, interacting may include grasping, cauterizing, expanding, applying energy, etc. The method may include removing a handle of the first endoluminal accessory while maintaining interaction with the first tissue portion. The method may include removing the first endoluminal instrument (e.g., an endoscope) while maintaining the interaction with the first tissue portion. The method may include reinserting the first endoluminal instrument parallel to the first endoluminal accessory and inserting at least a portion of a second endoluminal accessory into the working channel. The method may optionally include attaching the handle to the first endoluminal accessory or the second endoluminal accessory and performing a procedure with the second endoluminal accessory. The method functions to reversibly attach a handle to an accessory compatible with an instrument having a working channel (e.g., endoscope, catheter, sheath, trocar, etc.). The method may be used for endoscopy procedures, thrombectomy procedures, ligation procedures, ablation procedures, ultrasound procedures, electrocautery procedures, etc.

In some embodiments, the method 900 may include inserting at least a portion of a first endoluminal accessory into a lumen defined by a first endoluminal instrument in block S902, interacting with a first tissue portion with the first endoluminal accessory in block S904, removing a handle of the first endoluminal accessory while maintaining the interaction between the first endoluminal accessory and the first tissue portion in block S906, removing the first endoluminal instrument while maintaining the interaction between the first accessory and the first tissue portion in block S908, reinserting the first endoluminal instrument parallel to the first endoluminal accessory in block S910, inserting at least a portion of a second endoluminal accessory into the lumen defined by the first endoluminal instrument in block S912, optionally attaching the handle to the first endoluminal accessory or the second endoluminal accessory in block S914, and optionally performing a procedure with the second endoluminal accessory in block S916. The method functions to reversibly attach a handle to an accessory compatible with an endoscope or an endoscopy procedure. The method is used for endoscopy procedures but can additionally, or alternatively, be used for any suitable applications, clinical or otherwise. The method can be adapted to function for any suitable accessory where a removable handle is desirable.

In block S902, the method 900 includes inserting at least a portion of a first endoluminal accessory into a lumen defined by a first endoluminal instrument. In some embodiments, the at least a portion of the first endoluminal accessory may be inserted into a working channel of a first endoluminal instrument. In some embodiments, the first endoluminal instrument is an endoscope or an echoendoscope. The first endoluminal accessory may be an accessory (e.g., endoluminal accessory 112 of FIGS. 2A-2B, endoluminal accessory 612 of FIG. 9A) with a removable handle as described herein. In some embodiments, the first endoluminal accessory may be an accessory fitted with a removable handle (e.g., handle 770 of FIG. 10A) as described herein. In some embodiments, the first endoluminal accessory may be, but is not limited to, a miniature through-the-channel scope, a snare, a grasper, a forceps, a clip applier, a loop ligation accessory, an injection needle, a retrieval accessory, a retrieval basket, an electrosurgical knife, a sphincterotome, an extraction balloon, or a lithotripter. In some embodiments, the first endoluminal accessory may be a snare.

In block S904, the method 900 may include interacting with a first tissue portion with the first endoluminal accessory. In embodiments in which the first endoluminal accessory is a snare, the interacting with the first tissue portion may include grasping the first tissue portion such as by closing or tightening the snare. In embodiments in which the first endoluminal accessory is a scope, interacting with the first tissue portion may include repositioning the scope for a different view or illumination of the first tissue portion. In embodiments in which the first endoluminal accessory is a grasper, interacting with the first tissue portion may include a pinching or tightening of the grasper. In embodiments in which the first endoluminal accessory is a forceps, interacting with the first tissue portion may include a pinching or tightening of the forceps. In embodiments in which the first endoluminal accessory is an injection needle, interacting with the first tissue portion may include injecting at least a portion of a substance from the injection needle into the first tissue portion. In embodiments in which the first endoluminal accessory is an electrosurgical knife, interacting with the first tissue portion may include making an incision into the first tissue portion.

In block S906, the method 900 may include removing a handle of the first endoluminal accessory while maintaining the interaction between the first endoluminal accessory and the first tissue portion. In some embodiments, the handle is a removable handle (e.g., removable handle 100 of FIGS. 2A-2B, etc.) as described herein. In some embodiments, the removing of the handle may include placing a clamping portion (e.g., clamping portion 116 of FIGS. 2A-2B) in an unclamped state. For example, placing a clamping portion into an unclamped state may include pressing or releasing a spring-loaded button (e.g., button top 224 of FIG. 3A) or loosening a threaded structure (e.g., threaded structure 342 of FIG. 4A). In some embodiments, the removing of the handle may include positioning a coupling element (e.g., coupling element 114 of FIGS. 2A-2B) in an unlocked position. For example, positioning a coupling element into an unlocked position may include rotating a rotatable lock (e.g., rotatable lock 432 of FIGS. 5A-5B) or pressing or releasing an input element (e.g., the spring-loaded button as input element 552 of FIG. 8A). In some embodiments, lumen friction features, cable friction features (e.g., cable friction features 658 of FIG. 9A), adapter friction features (e.g., adapter friction features 779 of FIG. 10C) and/or friction receivers (e.g., friction receivers 783 of FIG. 10C) may at least substantially hold a cable (e.g., cable 656 of FIG. 9A) or a manipulatable element (e.g., manipulatable element 110 of FIGS. 2A-2B) in place (i.e., maintain a position) while the handle is being or has been removed and therefore may maintain the interaction with the first tissue portion.

In block S908, the method 900 may include removing the first endoluminal instrument (e.g., an endoscope) while maintaining the interaction between the first accessory and the first tissue portion. Because the first endoluminal instrument includes the working channel, in some embodiments, the first endoluminal instrument is removed over the working length of the first endoluminal accessory. This leaves the working length of the first endoluminal accessory exposed to the anatomy of the patient. In some embodiments, the removal of the first endoluminal instrument does not interfere or disturb (or does not substantially interfere or disturb) the operable state of the first endoluminal accessory.

In block S910, the method 900 may include reinserting the first endoluminal instrument parallel (e.g., at least partially adjacent to and towards the same target bodily region as) to the first endoluminal accessory. In some embodiments, reinserting the endoscope as the first endoluminal instrument parallel to the first endoluminal accessory provides restored vision of a bodily region during an operation.

In block S912, the method 900 may include inserting at least a portion of a second endoluminal accessory into the lumen defined by the first endoluminal instrument. As noted above, once the endoscope is withdrawn from over the first endoluminal accessory, it can be reinserted into the body alongside the first endoluminal accessory. At this point, a second endoluminal accessory may be introduced into the working channel of the endoscope. At least in some embodiments, two working channels may not be used together, except, for example, in cases where the endoscope has dual working channels, a second endoscope is employed, or an external auxiliary “working channel” (such as a medical tool used for suturing in endoscopic procedures) is utilized. In some embodiments, the second endoluminal accessory may be inserted directly into a patient's anatomy. In some embodiments, the second endoluminal accessory may be inserted into the working channel of the reinserted first endoluminal instrument. The second endoluminal accessory may be an accessory (e.g., endoluminal accessory 112 of FIGS. 2A-2B, endoluminal accessory 612 of FIG. 9A) with a removable handle as described herein. In some embodiments, the second endoluminal accessory may be an accessory fitted with a removable handle (e.g., handle 770 of FIG. 10A), as described herein. In some embodiments, the second endoluminal accessory may be, but is not limited to, a scope, a snare, a grasper, a par or forceps, a clip applier, a loop ligation accessory, an injection needle, a retrieval accessory, a retrieval basket, an electrosurgical knife, a sphincterotome, an extraction balloon, or a lithotripter. In some embodiments, the second endoluminal accessory may be a snare. In some embodiments, the second endoluminal accessory may be a scope.

In optional block S914, the method 900 may optionally include attaching the handle to the first endoluminal accessory or the second endoluminal accessory. In some embodiments, the attaching of the handle may include placing a clamping portion (e.g., clamping portion 116 of FIGS. 2A-2B) in a clamped state. For example, placing a clamping portion into a clamped state may include pressing or releasing a spring-loaded button (e.g., button top 224 of FIG. 3A) or tightening a threaded structure (e.g., threaded structure 342 of FIG. 4A). In some embodiments, the removing of the handle may include positioning a coupling element (e.g., coupling element 114 of FIGS. 2A-2B) in a locked position. For example, positioning a coupling element into a locked position may include rotating a rotatable lock (e.g., rotatable lock 432 of FIGS. 5A-5B) or pressing or releasing an input element (e.g., the spring-loaded button as input element 552 of FIG. 8A).

In block S916, the method 900 optionally includes performing a procedure with the second endoluminal accessory, in some embodiments. In some embodiments, performing a procedure with the second endoluminal accessory may include interacting with the first or a second tissue portion or performing another action. For example, in embodiments in which the second endoluminal accessory is a snare, the interacting with the first or second tissue portion may include grasping the first or second tissue portion such as by closing or tightening the snare. In embodiments in which the second endoluminal accessory is a grasper, interacting with the first or second tissue portion may include a pinching or tightening of the grasper. In embodiments in which the second endoluminal accessory is a forceps, interacting with the first or second tissue portion may include a pinching or tightening of the forceps. In embodiments in which the second endoluminal accessory is an injection needle, interacting with the first or second tissue portion may include injecting at least a portion of a substance from the injection needle into the first or second tissue portion. In embodiments in which the second endoluminal accessory is an electrosurgical knife, interacting with the first or second tissue portion may include making an incision into the first or second tissue portion.

### EXAMPLES

Embodiment 1. A removable handle for an endoscope compatible accessory, comprising: a shaft having: a first end opposite a second end, a groove configured to receive at least a portion of a manipulatable element of an endoluminal accessory, and a coupling element on the second end configured to reversibly couple to the endoluminal accessory; and a clamping portion configured to slide along the shaft and reversibly clamp to the manipulatable element of the endoluminal accessory, wherein a clamped state of the clamping portion is configured to allow manipulation of the manipulatable element by the clamping portion.

Embodiment 2. The removable handle of embodiment 1, wherein the clamping portion comprises one or more finger loops.

Embodiment 3. The removable handle of embodiment 1, wherein the clamping portion further defines a channel configured to receive an elongate body to clamp or unclamp the manipulatable element.

Embodiment 4. The removable handle of embodiment 3, wherein the elongate body further comprises an electrode for applying a current to a portion of the manipulatable element of the endoluminal accessory.

Embodiment 5. The removable handle of embodiment 3, wherein the elongate body comprises a screw.

Embodiment 6. The removable handle of embodiment 3, wherein the elongate body comprises a spring-loaded button.

Embodiment 7. The removable handle of embodiment 1, further comprising the endoluminal accessory.

Embodiment 8. The removable handle of embodiment 7, wherein the endoluminal accessory comprises a snare, grasper, a pair of forceps, a clip applier, a loop ligation accessory, an injection needle, a retrieval accessory, a retrieval basket, an electrosurgical knife, a sphincterotome, an extraction balloon, a lithotripter, or a stent.

Embodiment 9. The removable handle of embodiment 8, wherein the endoluminal accessory comprises a snare.

Embodiment 10. The removable handle of embodiment 9, wherein the manipulatable element comprises a wire such that sliding the clamping portion distally enlarges a loop in the wire and sliding the clamping portion proximally reduces the loop in the wire.

Embodiment 11. The removable handle of embodiment 1, wherein the clamped state is configured to allow advancement and retraction of the manipulatable element by the clamping portion.

Embodiment 12. The removable handle of embodiment 1, wherein the second end of the removable handle comprises: a channel configured to receive a proximal end of the endoluminal accessory; and the coupling element comprises a rotatable lock configured to be rotated about the shaft to secure the proximal end of the endoluminal accessory in the channel or release the proximal end of the endoluminal accessory from the channel.

Embodiment 13. The removable handle of embodiment 12, wherein the rotatable lock defines a lock channel having a reduced diameter section configured as a stop about at least a portion of a flange of the proximal end of the endoluminal accessory.

Embodiment 14. The removable handle of embodiment 13, wherein, in an unlocked state, the lock channel and the channel of the removable handle are aligned, and, in a locked state, the lock channel and the channel of the removable handle are offset.

Embodiment 15. The removable handle of embodiment 14, where offset comprises the lock channel being rotationally misaligned with the channel of the shaft.

Embodiment 16. The removable handle of embodiment 15, wherein the offset is between about 15 degrees to about 180 degrees.

Embodiment 17. The removable handle of embodiment 7, wherein a lumen of the endoluminal accessory comprises one or more friction features configured to reduce passive movement of the manipulatable element in the lumen of the endoluminal accessory.

Embodiment 18. The removable handle of embodiment 1, wherein the coupling element of the removable handle defines a variable diameter eyelet and comprises a spring-loaded button, wherein activation of the spring-loaded button is configured to cause interaction between a reduced diameter portion of the variable diameter eyelet with a flange of the endoluminal accessory to lock the endoluminal accessory in the second end, and de-activation of the spring-loaded button is configured to cause interaction between an enlarged diameter portion of the variable diameter eyelet with the flange of the endoluminal accessory to release the endoluminal accessory from the second end.

Embodiment 19. A method of removing a handle from an endoluminal accessory comprising: inserting at least a portion of a first endoluminal accessory into a patient; interacting with a first tissue portion with the first endoluminal accessory; and removing a handle of the first endoluminal accessory while maintaining the interacting of the first tissue portion.

Embodiment 20. The method of embodiment 19, wherein the at least a portion of the first endoluminal accessory is inserted into a working channel of a first endoluminal instrument in a patient.

Embodiment 21. The method of embodiment 20, wherein the first endoluminal instrument is an endoscope or a scope.

Embodiment 22. The method of embodiment 19, wherein the first endoluminal accessory is a grasper, a pair of forceps, a clip applier, a loop ligation accessory, an injection needle, a retrieval accessory, a retrieval basket, an electrosurgical knife, a sphincterotome, an extraction balloon, or a lithotripter.

Embodiment 23. The method of embodiment 19, wherein the interacting with the first the first tissue portion further comprises grasping the first tissue portion.

Embodiment 24. A method of accessing a bodily region with two or more endoluminal accessories, comprising: inserting at least a portion of a first endoluminal accessory into a lumen defined by a first endoluminal instrument; interacting with a first tissue portion with the first endoluminal accessory; removing a handle of the first endoluminal accessory while maintaining the interaction with the first tissue portion; removing the first endoluminal instrument while maintaining the interaction with the first tissue portion; reinserting the first endoluminal instrument parallel to the first endoluminal accessory; and inserting at least a portion of a second endoluminal accessory into the lumen defined by the first endoluminal instrument.

Embodiment 25. The method of embodiment 24, wherein the at least a portion of the first endoluminal accessory and the at least a portion of the second endoluminal accessory are inserted into a working channel of the first endoluminal instrument.

Embodiment 26. The method of embodiment 24, wherein one or both of: the first endoluminal accessory and the second endoluminal accessory are a snare, grasper, a pair of forceps, a clip applier, a loop ligation accessory, an injection needle, a retrieval accessory, a retrieval basket, an electrosurgical knife, a sphincterotome, an extraction balloon, or a lithotripter.

Embodiment 27. The method of embodiment 24, wherein the first endoluminal instrument is an endoscope or a scope.

Embodiment 28. The method of embodiment 24, further comprising attaching the handle to the first endoluminal accessory or the second endoluminal accessory.

Embodiment 29. The method of embodiment 24, further comprising performing a procedure with the second endoluminal accessory.

References in the specification to “one embodiment,” “an embodiment,” “an illustrative embodiment,” “some embodiments,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described.

As used in the description and claims, the singular form “a”, “an” and “the” include both singular and plural references unless the context clearly dictates otherwise. For example, the term “cable friction feature” may include, and is contemplated to include, a plurality of cable friction features. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (-) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A removable handle for an endoscope compatible accessory, comprising:
a shaft having:
a first end opposite a second end,
a groove configured to receive at least a portion of a manipulatable element of an endoluminal accessory, and
a coupling element on the second end configured to reversibly couple to the endoluminal accessory; and
a clamping portion configured to slide along the shaft and reversibly clamp to the manipulatable element of the endoluminal accessory,
wherein a clamped state of the clamping portion is configured to allow manipulation of the manipulatable element by the clamping portion.

2. The removable handle of claim 1, wherein the clamping portion comprises one or more finger loops.

3. The removable handle of claim 1, wherein the clamping portion further defines a channel configured to receive an elongate body to clamp or unclamp the manipulatable element.

4. The removable handle of claim 3, wherein the elongate body further comprises an electrode for applying a current to a portion of the manipulatable element of the endoluminal accessory.

5. The removable handle of claim 3, wherein the elongate body comprises a screw.

6. The removable handle of claim 3, wherein the elongate body comprises a spring-loaded button.

7. The removable handle of claim 1, further comprising the endoluminal accessory.

8. The removable handle of claim 7, wherein the endoluminal accessory comprises a snare, grasper, a pair of forceps, a clip applier, a loop ligation accessory, an injection needle, a retrieval accessory, a retrieval basket, an electrosurgical knife, a sphincterotome, an extraction balloon, a lithotripter, or a stent.

9. The removable handle of claim 8, wherein the endoluminal accessory comprises a snare.

10. The removable handle of claim 9, wherein the manipulatable element comprises a wire such that sliding the clamping portion distally enlarges a loop in the wire and sliding the clamping portion proximally reduces the loop in the wire.

11. The removable handle of claim 1, wherein the clamped state is configured to allow advancement and retraction of the manipulatable element by the clamping portion.

12. The removable handle of claim 1, wherein the second end of the removable handle comprises:
a channel configured to receive a proximal end of the endoluminal accessory; and
the coupling element comprises a rotatable lock configured to be rotated about the shaft to secure the proximal end of the endoluminal accessory in the channel or release the proximal end of the endoluminal accessory from the channel.

13. The removable handle of claim 12, wherein the rotatable lock defines a lock channel having a reduced diameter section configured as a stop about at least a portion of a flange of the proximal end of the endoluminal accessory.

14. The removable handle of claim 13, wherein, in an unlocked state, the lock channel and the channel of the removable handle are aligned, and, in a locked state, the lock channel and the channel of the removable handle are offset.

15. The removable handle of claim 14, where the offset comprises the lock channel being rotationally misaligned with the channel of the shaft.

16. The removable handle of claim 15, wherein the offset is between about 15 degrees to about 180 degrees.

17. The removable handle of claim 7, wherein a lumen of the endoluminal accessory comprises one or more friction features configured to reduce passive movement of the manipulatable element in the lumen of the endoluminal accessory.

18. The removable handle of claim 1, wherein the coupling element of the removable handle defines a variable diameter eyelet and comprises a spring-loaded button, wherein activation of the spring-loaded button is configured to cause interaction between a reduced diameter portion of the variable diameter eyelet with a flange of the endoluminal accessory to lock the endoluminal accessory in the second end, and de-activation of the spring-loaded button is configured to cause interaction between an enlarged diameter portion of the variable diameter eyelet with the flange of the endoluminal accessory to release the endoluminal accessory from the second end.

19. A method of removing a handle from an endoluminal accessory comprising:
inserting at least a portion of a first endoluminal accessory into a patient;
interacting with a first tissue portion with the first endoluminal accessory; and
removing a handle of the first endoluminal accessory while maintaining the interaction with the first tissue portion.

20. The method of claim 19, wherein the at least a portion of the first endoluminal accessory is inserted into a working channel of a first endoluminal instrument in a patient.

21. The method of claim 20, wherein the first endoluminal instrument is an endoscope or a echoendoscope.

22. The method of claim 19, wherein the first endoluminal accessory is a grasper, a pair of forceps, a clip applier, a loop ligation accessory, an injection needle, a retrieval accessory, a retrieval basket, an electrosurgical knife, a sphincterotome, an extraction balloon, or a lithotripter.

23. The method of claim 19, wherein the interaction with the first tissue portion further comprises grasping the first tissue portion.

24. A method of accessing a bodily region with two or more endoluminal accessories, comprising:
inserting at least a portion of a first endoluminal accessory into a lumen defined by a first endoluminal instrument;
interacting with a first tissue portion with the first endoluminal accessory;
removing a handle of the first endoluminal accessory while maintaining the interaction with the first tissue portion;
removing the first endoluminal instrument while maintaining the interaction with the first tissue portion;
reinserting the first endoluminal instrument parallel to the first endoluminal accessory; and
inserting at least a portion of a second endoluminal accessory into the lumen defined by the first endoluminal instrument.

25. The method of claim 24, wherein the at least a portion of the first endoluminal accessory and the at least a portion of the second endoluminal accessory are inserted into a working channel of the first endoluminal instrument.

26. The method of claim 24, wherein one or both of: the first endoluminal accessory and the second endoluminal accessory are a snare, grasper, a pair of forceps, a clip applier, a loop ligation accessory, an injection needle, a retrieval accessory, a retrieval basket, an electrosurgical knife, a sphincterotome, an extraction balloon, or a lithotripter.

27. The method of claim 24, wherein the first endoluminal instrument is an endoscope or a scope.

28. The method of claim 24, further comprising attaching the handle to the first endoluminal accessory or the second endoluminal accessory.

29. The method of claim 24, further comprising performing a procedure with the second endoluminal accessory.
